(19) United States
(12) Patent Application Publication
(10) Pub. No.: US 2004/0038891 A1
(43) Pub. Date: Feb. 26, 2004

(54) Methods and compositions for the treatment of ischemic reperfusion

(76) Inventors: Charles L. Bisgaier, Ann Arbor, MI (US); Michael E. Pape, Ann Arbor, MI (US); Jan Johansson, Milpitas, CA (US); Guido Franceschini, Milano (IT)

Correspondence Address: Pennie & Edmonds, LLP, 3300 Hillview Avenue, Palo Alto, CA, 94304, US

(21) Appl. No.: 10/440,214

(22) Filed: May 16, 2003

Related U.S. Application Data
(60) Provisional application No. 60/381,653, filed on May 17, 2002.
(60) Provisional application No. 60/405,478, filed on Aug. 23, 2002.

(51) Int. Cl. A61K 38/17; A61K 31/56

(52) U.S. Cl. 514/12; 514/171

(57) ABSTRACT

The invention provides methods and compositions for treating or preventing ischemic reperfusion injury. The methods of the instant invention comprise the administration of compositions comprising apolipoproteins, lecithin cholesterol acyltransferase or paraoxonase and lipid complexes thereof to treat, reduce or prevent ischemic reperfusion injury.

This application claims priority to U.S. Provisional Applications Serial Nos. 60/381,653 (filed May 17, 2002) and 60/405,478 (filed Aug. 23, 2002), each of which is incorporated herein by reference in its entirety.

### 1. TECHNICAL FIELD

The invention provides methods for treating, reducing or preventing ischemic reperfusion injury with compositions comprising apolipoproteins or apolipoprotein agonists.

### 2. BACKGROUND OF THE INVENTION

Ischemia followed by reperfusion is the major cause of skeletal and cardiac muscle damage in mammals. Ischemia is caused by a reduction in oxygen supplied to tissues or organs as a result of reduced blood flow and can lead to organ dysfunction. Reduced blood supply can result from occlusion or blood diversion due to vessel thrombosis, such as myocardial infarction, stenosis, accidental vessel injury, or surgical procedures. Subsequent reestablishment of an adequate supply of oxygenated blood to the tissue can result in increased damage, a process known as ischemia reperfusion injury or occlusion reperfusion injury. Complications arising from ischemia reperfusion injury include stroke, fatal or non-fatal myocardial infarction, myocardial remodeling, aneurysms, peripheral vascular disease, tissue necrosis, kidney failure, and post-surgical loss of muscle tone.

Ischemia can result secondary to occlusive events including stenosis, or thrombosis. Stenosis can result due to a medical condition such as atherosclerosis or induced during a surgical procedure. For example, surgical procedures (knee, hand, hip and shoulder surgery), tissue transplantation, cardiac procedures including coronary artery bypass graft (CABG) and percutaneous transluminal coronary angioplasty (PTCA) can all reduce or stop blood flow and induce ischemia and set the stage for reperfusion injury. Furthermore, harvested donor tissue and organs are also susceptible to reperfusion injury while in transit and following transplantation in a recipient.

Oxygen free radicals are considered to be important components involved in the pathophysiology of ischemia/reperfusion. (See, Banerjee et al., 2002, BMC Pharmacol 2(1):16; Demir and Inal-Erden 1998, Clin. Chim. Acta 275(2): 127-35; Fukuzawa et al., 1995, Transplantation 58(1):6-9; Sewerynek et al., 1996, Hepatogastroenterology 43(10):898-905; Serteser et al., 2002, J. Surg. Res. 107(2) 234-40).

In animal models, reactive oxygen species have been shown to be involved in reperfusion injury to a variety of tissues and organs, including the kidneys, brain, liver and heart. (Sener et al., 2002 , J. Pineal Res. 32(2):120-6; Sener et al., 2003, Life Sci. 72(24):2707-18; Ding-Zhou et al., 2003, J. Pharmacol. Exp. Ther. [e-publication ahead of print May, 2, 2003] PMID: 12730357; Katamaya et al., 1997, Tokai J. Exp. Clin. Med. 22(2):33-44; Grech et al., 1996, Am. J. Cardiol. 77(2):122-7).

In humans, reactive oxygen species are also thought to mediate ischemia reperfusion injury. The enzyme xanthine oxidase is responsible for the release of oxygen free radicals during myocardial reperfusion (See Guan et al., 1999, Jpn. Cir. J 63(12):924-8). Pretreatment with allopurinol, a xanthine oxidase inhibitor, for patients undergoing coronary artery surgery or PTCA after acute myocardial infarction provided improved cardiac health. For example, patients pretreated with allopurinol showed decreased episodes of arrhythmia and improved left ventricular function when compared to the control group (Bochenek et al., 1990, Eur. J. Cardiothorac. Surg. 4(10):538-42; Guan et al., 2003, J. Cardiovas. Pharmacol 41(5):699-705).

Ischemia injury can also occur due to the release of pro-inflammatory cytokines, chemokines and other mediators such as tumor necrosis factor, interleukins and interferons from epithelial and endothelial cells. (Furuicha et al., 2002, Drug News Perspect. 15(8):477-82; Donnahoo et al., 1999, J. Urol. 162(1):196-203; Yoshimoto et al., 1997, Acta Neuropathol. (Berl) 93(2): 154-8; Sung et al., 2002, Kidney Int. 62(4):1160-7; Maekawa et al., 2002, J. Am. Coll. Cardiol. 39(7):1229-35). The release of cytokines, in turn, attracts a multitude of cells such as leukocytes, including neutrophils, monocytes and macrophages which contribute to an inflammatory cascade (Taub 1996, Cytokine Growth Factor Rev. 7(4):355-76; Krishnadasan et al., 2003, J. Thorac. Cardiovasc. Surg. 125(2):261-72; Krishnaswamy et al., 1999, J. Interferon Cytokine Res. 19(2):91-104; Sener et al., 2003, Life Sci. 73(1):81-91; Yue, et al., 2001, Circulation 104(21):2588-94).

A variety of drugs have been studied as potentially effective agents in the treatment or prevention of ischemia reperfusion injury, including, pentoxifylline, N-acetylcysteine, garlic, melatonin, vitamin C and BN 80933 (a neuronal nitric oxide synthase inhibitor and antioxidant) with limited success (Demir and Inal-Erden 1998, Clin. Chim. Acta 275(2):127-35; Banerjee et al., 2002, BMC Pharmacol. 2(1):16; Fukuzawa et al., 1995, Transplantation 58(1):6-9; Sener et al., 2002, J. Pineal Res. 32(2):120-6; Ding-Zhou et al., 2003, J. Pharmacol. Exp. Ther. [e-publication ahead of print May, 2, 2003] PMID: 12730357; Guan et al., 1999, Jpn. Cir. J 63(12):924-8).

Current ischemia therapy focuses on restoring blood flow as quickly as possible. Rapid treatment following, for example, acute myocardial infarction (AMI), is vital to preventing long term injury. Thrombolytic treatment more than 24 hours after the onset of AMI does not improve clinical outcome. The use of PTCA to revascularize after AMI remains controversial but studies indicate that PTCA performed within 48 hours after AMI is beneficial. Noted benefits include, for example, preventing left ventricular remodeling, decreasing left ventricular remodeling and ananeurysm, improving left ventricular wall motion and decreasing cardiac events for a 5 year period after an AMI. (Kanamasa et al., 2000, J. Thromb. Thrombolysis 9(1):47-51; Kanamasa et al., 1996, J. Cardiol. 28(4):199-205; Horie et al., 1998, Circulation 98(22):2377-82; Kanamasa et al., 2000, Angiology 51(4):281-8).

Although thrombolytic therapy and PTCA are used for reperfusion, both have significant drawbacks. For example, thrombolytic therapy is contraindicated in patients with active internal bleeding, a history of cerebrovascular accidents, intracranial or intraspinal surgery or trauma, arteriovenus malformation or aneurysm, intracranial neoplasm, bleeding diathesis and severe uncontrolled hypertension (Drug Facts and Comparisons, updated monthly, January 2003, Facts and Comparisons, Wolter Kluwer Company., St. Louis, Mo.). PTCA is an invasive procedure and carries its own set of risks including death, myocardial infarction and stroke, and is relatively contraindicated in patients with preexisting poor cardiac health and coagulopathies (The Merck Manual, 17^{th} Ed. (Beers and Berkow, Eds.) Merck Research Laboratories, Whitehouse Station, N.J., 1999, p.1628-9).

Even when thrombolytics or PTCA can be used in a patient in need of reperfusion, neither acts to treat or prevent ischemic reperfusion injury. New methods and compositions are needed to treat or ameliorate the symptoms of ischemic reperfusion injury.

### 3. SUMMARY OF THE INVENTION

Accordingly, the invention provides methods and compositions for treating, reducing or preventing ischemic reperfusion injury. The methods provide for treating, reducing or preventing ischemic reperfusion injury using compositions comprising apolipoproteins, lecithin cholesterol acyltransferase or paroxonase. The methods of the instant invention comprise the administration of ischemic reperfusion injury agents of the invention. Surprisingly, it has been discovered that administration of ischemic reperfusion injury agents can treat, reduce or protect an individual from ischemic reperfusion injury.

In one aspect, the present invention provides methods of treating, reducing or preventing ischemic reperfusion injury by administration of an effective amount of an ischemic reperfusion injury agent. In certain embodiments of the invention, the agent can be an apolipoprotein, lecithin cholesterol acyltransferase or paraoxonase. In particular embodiments, the ischemic reperfusion agent is an apolipoprotein. The apolipoprotein can be any apolipoprotein including, for example, apolipoprotein A-I or a variant or fragment thereof. In certain embodiments the apolipoprotein is a thiol containing apolipoprotein. In preferred embodiments of the invention, the apolipoprotein is apolipoprotein A-I Milano (ApoA-I_{M}).

In certain embodiments, the ischemic reperfusion agent can be administered in the form of a complex comprising an apolipoprotein and a lipid. Preferably, the lipid is a phospholipid. The phospholipid can be any phospholipid known to those of skill in the art. In preferred embodiments of the invention the phospholipid can be phosphatidylcholine or a derivative or analogue thereof such as 1-palmitoyl-2-oleoyl phosphatidylcholine.

The methods and compositions of the invention can be useful in any context where treatment, reduction or protection from ischemic reperfusion injury might be useful. In certain embodiments, the methods and compositions of the invention can protect the muscle and organs such as, for example, the heart, liver, kidney, brain, lung, spleen and steroidogenic organs (e.g., thyroid, adrenal glands and gonads) from damage as a result of ischemia reperfusion injury. In certain embodiments, the methods and compositions of the invention can protect tissues, muscles or organs during transplantation harvesting, transit and implantation into a transplant recipient.

### 4. DESCRIPTION OF THE FIGURES

FIG. 1 provides a diagram of two apolipoprotein A-I Milano chains;

FIG. 2 provides a diagram of a Langendorff Apparatus to treat ex vivo and monitor cardiac function in the isolated rabbit heart;

FIG. 3 provides a closer view of the heart as mounted in the Langendorff Apparatus;

FIG. 4 provides an example of a protocol wherein isolated hearts were treated with vehicle or ETC-216 prior to the onset of ischemia;

FIG. 5 provides creatine kinase activity in coronary venous effluent;

FIG. 6 provides real-time monitoring of cardiac function collected from a vehicle and an ETC-216 treated isolated rabbit heart in the Langendorff Apparatus;

FIG. 7 provides the temporal changes in left ventricular developed pressure (LVDP) in isolated rabbit hearts before, during and after 30 minutes of global ischemic arrest and 60 minutes of reperfusion;

FIG. 8 provides temporal changes in left ventricular end-diastolic pressure (LVEDP) in isolated rabbit hearts before, during and after 30 minutes of global ischemic arrest and 60 minutes of reperfusion;

FIG. 9 provides temporal changes in coronary perfusion pressure (CPP) in isolated rabbit hearts before, during and after 30 minutes of global ischemic arrest and 60 minutes of reperfusion;

FIG. 10 provides lipid hydroperoxide content in tissue homogenates from vehicle and ETC-216 treated rabbit hearts subjected to global ischemic arrest for 30 minutes followed by 60 minutes reperfusion;

FIG. 11 provides electron microscope images of cardiac muscle samples from vehicle and ETC-216 treated rabbit hearts;

FIG. 12 provides an additional protocol of the present invention wherein one pretreatment was administered prior to the onset of ischemia in the acute administration group and two pretreatments were administered prior to the onset of ischemia in the chronic administration group;

FIG. 13 provides a protocol for determination of infarct size;

FIG. 14 provides infarct percent of area at risk, infarct percent of left ventricle, and area at risk percent of left ventricle in rabbits treated once (i.e., acute treatment) or treated twice (i.e., chronic treatment) with ETC-216 (100 mg/kg) or an equivalent volume of vehicle;

FIG. 15 provides an additional protocol of the present invention wherein rabbits were pretreated prior to the onset of ischemia with either vehicle (Group 1) or 10, 3 or 1 mg/kg of ETC-216 (Group 2);

FIG. 16 provides infarct percent of area at risk, infarct percent of left ventricle, and area at risk percent of left ventricle determined in rabbits treated once (i.e., acute treatment) with 10, 3 or 1 mg/kg of ETC-216 or with an equivalent volume of sucrose-mannitol vehicle for each group;

FIG. 17 provides temporal changes in lipoprotein unesterified cholesterol;

FIG. 18 provides an additional protocol of the present invention wherein a single treatment of vehicle of ETC-216 was administered during the last 5 minutes of the 30 minute ischemic period; and

FIG. 19 provides infarct percent of area at risk, infarct percent of left ventricle, and area at risk percent of left ventricle determined in rabbits.

### 5. DETAILED DESCRIPTION OF THE INVENTION

The invention provides methods of treating, reducing or preventing ischemic reperfusion injury with a preventative reperfusion injury agent. The agent can be any preventative ischemic reperfusion injury agent described herein including, for example, an apolipoprotein, lecithin cholesterol acyltransferase or paraoxonase.

5.1. Apolipoprotein

In one aspect, the present invention provides methods for the treatment, reduction or prevention of injury from ischemic reperfusion by administering a composition comprising an apolipoprotein. As used herein, the term “apolipoprotein” refers to apolipoproteins known to those of skill in the art and variants and fragments thereof and to apolipoprotein agonists, analogues or fragments thereof described below.

The apolipoprotein can be any apolipoprotein that is effective for the treatment or prevention of injury from ischemic reperfusion. Suitable apolipoproteins include, but are not limited to, ApoA-I, ApoA-II, ApoA-IV, ApoA-V and ApoE, and active polymorphic forms, isoforms, variants and mutants as well as fragments or truncated forms thereof. In certain embodiments, the apolipoprotein is a thiol containing apolipoprotein. “Thiol containing apolipoprotein” refers to an apolipoprotein, variant, fragment or isoform that contains at least one cysteine residue. The most common thiol containing apolipoproteins are ApoA-I Milano (ApoA-I_{M}) and ApoA-I Paris (ApoA-I_{P}) which contain one cysteine residue (Jia et al., 2002, Biochem. Biophys. Res. Comm. 297: 206-13; Bielicki and Oda, 2002, Biochemistry 41: 2089-96). ApoA-II, ApoE2 and ApoE3 are also thiol containing apolipoproteins. In certain embodiments, the apolipoprotein is not a thiol containing apolipoprotein, such as ApoA-I.

In certain embodiments, the apolipoprotein can be in its mature form, in its preproapolipoprotein form or in its proapolipoprotein form. Homo- and heterodimers (where feasible) of pro- and mature ApoA-I (Duverger et al., 1996, Arterioscler. Thromb. Vasc. Biol. 16(12):1424-29), ApoA-I Milano (Klon et al., 2000, Biophys. J. 79:(3)1679-87; Franceschini et al., 1985, J. Biol. Chem. 260: 1632-35), ApoA-I Paris (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), and ApoE (McLean et al., 1983, J. Biol. Chem. 258(14):8993-9000) can also be utilized within the scope of the invention.

In certain embodiments, the apolipoprotein can be a fragment, variant or isoform of the apolipoprotein. The term “fragment” refers to any apolipoprotein having an amino acid sequence shorter than that of a native apolipoprotein and which fragment retains the activity of native apolipoprotein, including lipid binding properties. By “variant” is meant substitutions or alterations in the amino acid sequences of the apolipoprotein, which substitutions or alterations, e.g., additions and deletions of amino acid residues, do not abolish the activity of native apolipoprotein, including lipid binding properties. Thus, a variant can comprise a protein or peptide having a substantially identical amino acid sequence to a native apolipoprotein provided herein in which one or more amino acid residues have been conservatively substituted with chemically similar amino acids. Examples of conservative substitutions include the substitution of at least one hydrophobic residue such as isoleucine, valine, leucine or methionine for another. Likewise, the present invention contemplates, for example, the substitution of at least one hydrophilic residue such as, for example, between arginine and lysine, between glutamine and asparagine, and between glycine and serine (see U.S. Pat. Nos. 6,004,925, 6,037,323 and 6,046,166). The term “isoform” refers to a protein having the same, greater or partial function and similar, identical or partial sequence, and may or may not be the product of the same gene and usually tissue specific (see Weisgraber 1990, J. Lipid Res. 31(8):1503-11; Hixson and Powers 1991, J. Lipid Res. 32(9):1529-35; Lackner et al., 1985, J. Biol. Chem. 260(2):703-6; Hoeg et al., 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vase. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sacre et al., 2003, FEBS Lett. 540(1-3):181-7; Weers, et al., 2003, Biophys. Chem. 100(1-3):481-92; Gong et al., 2002, J. Biol. Chem. 277(33):29919-26; Ohta et al., 1984, J. Biol. Chem. 259(23):14888-93 and U.S. Pat. No. 6,372,886). In certain embodiments, the methods and compositions of the present invention include the use of a chimeric construction of an apolipoprotein. For example, a chimeric construction of an apolipoprotein can be comprised of an apolipoprotein domain with high lipid binding capacity associated with an apolipoprotein domain containing ischemia reperfusion protective properties. A chimeric construction of an apolipoprotein can be a construction that includes separate regions within an apolipoprotein (i.e., homologous construction) or a chimeric construction can be a construction that includes separate regions between different apolipoproteins (i.e., heterologous constructions). Compositions comprising a chimeric construction can also include segments that are apolipoprotein variants or segments designed to have a specific character (e.g., lipid binding, receptor binding, enzymatic, enzyme activating, antioxidant or reduction-oxidation property) (see Weisgraber 1990, J. Lipid Res. 31(8):1503-11; Hixson and Powers 1991, J. Lipid Res. 32(9):1529-35; Lackner et al., 1985, J. Biol. Chem. 260(2):703-6; Hoeg et al, 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vasc. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sorenson et al., 1999, Arterioscler. Thromb. Vasc. Biol. 19(9):2214-25; Palgunachari 1996, Arterioscler. Throb. Vasc. Biol. 16(2):328-38: Thurberg et al., J. Biol. Chem. 271(11):6062-70; Dyer 1991, J. Biol. Chem. 266(23):150009-15; Hill 1998, J. Biol. Chem. 273(47):30979-84).

Apolipoproteins utilized in the invention also include recombinant, synthetic, semi-synthetic or purified apolipoproteins. Methods for obtaining apolipoproteins or equivalents thereof, utilized by the invention are well-known in the art. For example, apolipoproteins can be separated from plasma or natural products by, for example, density gradient centrifugation or immunoaffinity chromatography, or produced synthetically, semi-synthetically or using recombinant DNA techniques known to those of the art (see, e.g., Mulugeta et al., 1998, J. Chromatogr. 798(1-2): 83-90; Chung et al., 1980, J. Lipid Res. 21(3):284-91; Cheung et al., 1987, J. Lipid Res. 28(8):913-29; Persson, et al., 1998, J. Chromatogr. 711:97-109; U.S. Pat. Nos. 5,059,528, 5,834,596, 5,876,968 and 5,721,114; and PCT Publications WO 86/04920 and WO 87/02062).

Apolipoproteins utilized in the invention further include apolipoprotein agonists such as peptides and peptide analogues that mimic the activity of ApoA-I, ApoA-I Milano (ApoA-I_{M}), ApoA-I Paris (ApoA-I_{P}), ApoA-II, ApoA-IV, and ApoE. For example, the apolipoprotein can be any of those described in U.S. Pat. Nos. 6,004,925, 6,037,323, 6,046,166, and 5,840,688, the contents of which are incorporated herein by reference in their entireties.

Apolipoprotein agonist peptides or peptide analogues can be synthesized or manufactured using any technique for peptide synthesis known in the art including, e.g., the techniques described in U.S. Pat. Nos. 6,004,925, 6,037,323 and 6,046,166. For example, the peptides may be prepared using the solid-phase synthetic technique initially described by Merrifield (1963, J. Am. Chem. Soc. 85:2149-2154). Other peptide synthesis techniques may be found in Bodanszky et al., Peptide Synthesis, John Wiley & Sons, 2d Ed., (1976) and other references readily available to those skilled in the art. A summary of polypeptide synthesis techniques can be found in Stuart and Young, Solid Phase Peptide. Synthesis, Pierce Chemical Company, Rockford, Ill., (1984). Peptides may also be synthesized by solution methods as described in The Proteins, Vol. II, 3d Ed., Neurath et. al., Eds., p. 105-237, Academic Press, New York, N.Y. (1976). Appropriate protective groups for use in different peptide syntheses are described in the above-mentioned texts as well as in McOmie, Protective Groups in Organic Chemistry, Plenum Press, New York, N.Y. (1973). The peptides of the present invention might also be prepared by chemical or enzymatic cleavage from larger portions of, for example, apolipoprotein A-I.

In certain embodiments, the apolipoprotein can be a mixture of apolipoproteins. In one embodiment, the apolipoprotein can be a homogeneous mixture, that is, a single type of apolipoprotein. In another embodiment, the apolipoprotein can be a heterogeneous mixture of apolipoproteins, that is, a mixture of two or more different apolipoproteins. Embodiments of heterogenous mixtures of apolipoproteins can comprise, for example, a mixture of an apolipoprotein from an animal source and an apolipoprotein from a semi-synthetic source. In certain embodiments, a heterogenous mixture can comprise, for example, a mixture of ApoA-I and ApoA-I Milano. In certain embodiments, a heterogeneous mixture can comprise, for example, a mixture of ApoA-I Milano and ApoA-I Paris. Suitable mixtures for use in the methods and compositions of the invention will be apparent to one of skill in the art.

If the apolipoprotein is obtained from natural sources, it can be obtained from a plant or animal source. If the apolipoprotein is obtained from an animal source, the apolipoprotein can be from any species. In certain embodiments, the apolipoprotien can be obtained from an animal source. In certain embodiments, the apolipoprotein can be obtained from a human source. In preferred embodiments of the invention, the apolipoprotein is derived from the same species as the individual to which the apolipoprotein is administered.

5.2. Lecithin Cholesterol Acyltransferase

In another aspect, the present invention provides methods for the treatment, reduction or prevention of injury from ischemic reperfusion by administering a composition comprising lecithin cholesterol acyltransferase (LCAT). As used herein, the term “LCAT” refers to the enzyme that catalyzes the transacyation of lecithin known to those of skill in the art and variants and fragments thereof (see, Jauhiainen et al, 1988, J. Biol. Chem. 263(14):6525-33; S. Pat. No. 6,498,019 the contents of which are incorporated herein by reference in their entireties).

The LCAT can be any LCAT that is effective for the treatment or prevention of injury from ischemic reperfusion. The LCAT utilized by the invention also include recombinant or purified LCAT. Methods for obtaining LCAT or equivalents thereof, utilized by the invention are well-known in the art (see, Jauhiainen et al., 1988, J. Biol. Chem. 263(14):6525-33; Vakkilainen et al., 2002, J. Lipid Res. 43(4):598-603; Jiang et al., 1999, J. Clin. Invest. 103(6):907-14; Lee, et al., 2003, J. Biol. Chem. 278(15):13539-45; Gambert 1995, C. R. Seances Soc. Biol. Fil. (article in French) 189(5):883-8; Jonas 2000, Biochim. Biophys. Acta 1529(1-3):245-56; U.S. Pat. No. 6,498,019 the contents of which are incorporated herein by reference in their entireties).

5.3. Paraoxonase

In another aspect, the present invention provides methods for the treatment, reduction or prevention of injury from ischemic reperfusion by administering a composition comprising paraoxonase. As used herein, “paraoxonase” refers to the enzyme originally found to be responsible for the hydrolysis of paraoxon and is physically associated with an apolipoprotein (ApoA-I) and clusterin-containing high-density lipoprotein and prevents low-density lipoprotein from lipid peroxidation (Laplaud et al., 1998, Clin. Chem. Lab. Med. 36(7):431-41; Paragh et al., 1998, Nephron 81(2):166-70; Ayub et al., 1999 Arterioscler. Thromb. Vasc. Biol. 19(2):330-5; Tanimoto et al., 2003, Life Sci. 72(25):2877-85; U.S. Pat. Nos. 6,521,226, 6,391,298 and 6,242,186).

5.4. Phospholipid Complexes

In certain embodiments of the invention, the apolipoprotein, lecithin cholesterol acyltransferase or paraoxonase can be administered in a complex comprising a lipid and the apolipoprotein, lecithin cholesterol acyltransferase or paraoxonase. The lipid can be any lipid known to those of skill in the art. In certain embodiments of the invention, the lipid is a phospholipid.

The phospholipid can be obtained from any source known to those of skill in the art. For example, the phospholipid can be obtained from commercial sources, natural sources or by synthetic or semi-synthetic means known to those of skill in the art (Melńichuk et al., 1987, Ukr. Biokhim. Zh. 59(6):75-7; Melńichuk et al., 1987, Ukr. Biokhim. Zh. 59(5):66-70; Ramesh et al., 1979, J. Am. Oil Chem. Soc. 56(5):585-7; Patel and Sparrow, 1978, J. Chromatogr. 150(2):542-7; Kaduce et al., 1983, J. Lipid Res. 24(10):1398-403; Schlueter et al., 2003, Org. Lett. 5(3):255-7; Tsuji et al., 2002, Nippon Yakurigaku Zasshi 120(1):67P-69P).

The phospholipid can be any phospholipid known to those of skill in the art. For example, the phospholipid can be a small alkyl chain phospholipid, phosphatidylcholine, egg phosphatidylcholine, soybean phosphatidylcholine, dipalmitoylphosphatidylcholine, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, (1,3)-D-mannosyl-(1,3)diglyceride, aminophenylglycoside, 3-cholesteryl-6′-(glycosylthio)hexyl ether glycolipids, and cholesterol and its derivatives. The phospholipid can also be derivatives or analogues of any of the above phospholipids. In certain embodiments, the composition can comprise combinations of two or more phospholipids. In preferred embodiments of the invention, the apolipoprotein is administered in a complex with 1-palmitoyl-2-oleoyl phosphatidylcholine (“POPC”). In a preferred embodiment of the invention, the apolipoprotein is a recombinant apolipoprotein A-I Milano (ApoA-I Milano) complexed with l-palmitoyl-2-oleoyl phosphatidylcholine in a one to one ratio by weight. This complex is referred to as ETC-216.

The compositions can comprise any amount of phospholipid and any amount of apolipoprotein, lecithin cholesterol acyltransferase or paraoxonase effective to treat or prevent injury from ischemic reperfusion. In certain embodiments, the composition can comprise a complex of an apolipoprotein and a phospholipid in a ratio of about one to about one. However, the compositions can comprise complexes with other ratios of phospholipid to apolipoprotein such as about 100:1, about 10:1, about 5:1, about 3:1, about 2:1, about 1:1, about 1:2, about 1:3, about 1:5, about 1:10 and about 1:100. Optimization of the ratio of phospholipid to apolipoprotein is within the skill of those in the art.

5.5. Methods of Making Lipid Complexes

In one aspect, the present invention provides methods for the treatment, reduction or prevention of injury from ischemic reperfusion by administering a composition comprising an apolipoprotein, lecithin cholesterol acyltransferase or paraoxonase complexed with a lipid. In one embodiment, the composition is comprised of an apolipoprotein-lipid complex.

A complex comprising an apolipoprotein and a lipid can be prepared in a variety of forms, including, but not limited to vesicles, liposomes or proteoliposomes. A variety of methods well known to those skilled in the art can be used to prepare the complex comprising an apolipoprotein and a lipid (an apolipoprotein-lipid complex). A number of available techniques for preparing liposomes or proteoliposomes may be used. For example, an apolipoprotein can be co-sonicated (using a bath or probe sonicator) with the appropriate lipid to form complexes. Alternatively, apolipoprotein can be combined with preformed lipid vesicles resulting in the spontaneous formation of a complex comprising an apolipoprotein and a lipid. The apolipoprotein-lipid complexes can also be formed by a detergent dialysis method; e.g., a mixture of apolipoprotein, lipid and a detergent such as cholate can be dialyzed to remove the detergent and reconstituted to form apolipoprotein-lipid complexes (see e.g., Jonas et al., 1986, Methods Enzymol. 128, 553-82), or by using an extruder device or by homogenization. Other methods are disclosed, for example, in U.S. Pat. Nos. 6,004,925, 6,037,323 and 6,046,166, incorporated herein by reference in their entireties. Exemplary methods of preparing apolipoprotein lipid complexes by co-lyophilization are described in U.S. Pat. No. 6,287,590, the content of which is hereby incorporated by reference in its entirety. Other methods of preparing apolipoprotein-lipid complexes will be apparent to those of skill in the art.

In certain embodiments, the complex comprises lecithin cholesterol acyltransferase and a lipid. In another embodiment, the complex comprises paraoxonase and a lipid.

5.6. Pharmaceutical Formulations

The invention provides methods and compositions useful for treating, reducing or preventing or ischemia reperfusion injury. In certain embodiments, the compositions of the invention are pharmaceutical compositions. In one embodiment, the pharmaceutical composition comprises an apolipoprotein, lecithin cholesterol acyltransferase or paraoxonase and a lipid in a pharmaceutically acceptable composition. A pharmaceutically acceptable composition, as will be described, below, includes, for example, an acceptable diluent, excipient or carrier.

In preferred embodiments, the pharmaceutical compositions comprise an apolipoprotein. In another preferred embodiment, the pharmaceutical compositions comprise an apolipoprotein-lipid complex. For the purposes of this section of the application, the term “apolipoprotein” refers either to an apolipoprotein or to a composition comprising a complex of an apolipoprotein and a lipid (“apolipoprotein-lipid complex”).

The pharmaceutical compositions of the present invention comprise the apolipoprotein, lecithin cholesterol acyltransferase or paraoxonase in a pharmaceutically acceptable composition suitable for administration and delivery in vivo or to an extracorporeal (ex vivo) tissue or organ.

The pharmaceutical compositions can comprise the apolipoprotein, lecithin cholesterol acyltransferase or paraoxonase in a salt form. For example, because proteins can comprise acidic and/or basic termini and/or side chains, the proteins can be included in the pharmaceutical compositions in either the form of free acids or bases, or in the form of pharmaceutically acceptable salts. Pharmaceutically acceptable salts can include, suitable acids which are capable of forming salts with the proteins of the present invention including, for example, inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with the subject proteins can include, for example, inorganic bases such as sodium hydroxide, ammonium hydroxide, potassium hydroxide and the like; and organic bases such as mono-, di-and tri-alkyl amines (e.g., triethyl amine, diisopropyl amine, methyl amine, dimethyl amine and the like) and optionally substituted ethanolamines (e.g., ethanolamine, diethanolamine and the like).

The pharmaceutical composition can be in a variety of forms suitable for any route of administration, including, but not limited to, parenteral, enteral, topical or inhalation. Parenteral administration refers to any route of administration that is not through the alimentary canal, including, but not limited to, injectable administration (i.e., intravenous, intramuscular and the like as described below). Enteral administration refers to any route of administration which is oral, including, but not limited to, tablets, capsules, oral solutions, suspensions, sprays and the like, as described below. For purposes of this section, enteral administration also refers to rectal and vaginal routes of administration. Topical administration refers to any route of administration through the skin, including, but not limited to, creams, ointments, gels and transdermal patches, as described below (see also, Remington's Pharmaceutical Sciences, 18^{th} Edition Gennaro et al., eds.) Mack Printing Company, Easton, Pa., 1990).

Parenteral pharmaceutical compositions of the present invention can be administered by injection, for example, into a vein (intravenously), an artery (intraarterially), a muscle (intramuscularly), under the skin (subcutaneously or in a depot composition), to the pericardium, to the coronary arteries, or used as a solution for delivery to a tissue or organ (for example, use in a cardiopulmonary bypass machine or to bathe transplant tissues or organs, as described below).

Injectable pharmaceutical compositions can be sterile suspensions, solutions or emulsions of the apolipoprotein, lecithin cholesterol acyltransferase or paraoxonase or lipid complexes thereof in aqueous or oily vehicles. The compositions may also comprise formulating agents or excipients, such as suspending, stabilizing and/or dispersing agents. The formulations for injection may be presented in unit dosage form, e.g., in ampules or in multidose containers, and may comprise added preservatives. In certain embodiments, the pharmaceutical compositions contain buffers such as tris(hydroxymethyl)aminomethane or THAM (tromethamine).

Injectable compositions can be pharmaceutically appropriate compositions for any route of injectable administration, including, but not limited to, intravenous, intrarterial, intracoronary, pericardial, perivascular, intramuscular, subcutaneous and intraarticular. The injectable pharmaceutical compositions can be a pharmaceutically appropriate composition for administration directly into the heart, pericardium or coronary arteries.

The parenteral pharmaceutical compositions can be pharmaceutically appropriate compositions suitable for bathing transplantation tissue or organs before, during or after transit to the intended recipient. Such compositions can be used before or during preparation of the tissue or organ for transplant (e.g., before or during harvesting). In addition, the preparation can be a cardioplegic solution administered during cardiac surgery. In certain embodiments, the pharmaceutical composition can be used, for example, in conjunction with a cardiopulmonary bypass machine to provide the pharmaceutical composition to the heart. Such preparations can be used during the induction, maintenance or reperfusion stages of cardiac surgery (see Chang et al., 2003, Masui 52(4):356-62; Ibrahim et al., 1999, Eur. J. Cardiothorac. Surg. 15(1):75-83; von Oppell et al, 1991, J. Thorac. Cardiovasc. Surg. 102(3):405-12; Ji et al., 2002, J. Extra Corpor. Technol. 34(2):107-10). In certain embodiments, the pharmaceutical composition can be delivered via a mechanical device such as a pump or perfuser (e.g. PerDUCER®) (Hou and March 2003, J. Invasive Cardiol. 15(1):13-7; Maisch et al., 2001, Am. J. Cardiol. 88(11):1323-6; Macris and Igo 1999, Clin. Cardiol. 22(1, Suppl 1): 136-9).

Alternatively, the injectable pharmaceutical composition can be provided in powder form for reconstitution with a suitable vehicle, including but not limited to sterile pyrogen free water, buffer, dextrose solution, etc., before use. To this end, the apolipoprotein, lecithin cholesterol acyltransferase or paraoxonase can be lyophilized, or co-lyophilized with a lipid, as appropriate. The pharmaceutical compositions can be supplied in unit dosage forms and reconstituted prior to use in vivo. Methods of preparing apolipoprotein lipid complexes by co-lyophilization are described, for example, in U.S. Pat. No. 6,287,590, the content of which is hereby incorporated by reference in its entirety.

For prolonged delivery, the pharmaceutical composition can be provided as a depot preparation, for administration by implantation; e.g., subcutaneous, intradermal, or intramuscular injection. Thus, for example, the pharmaceutical composition can be formulated with suitable polymeric or hydrophobic materials (e.g., as an emulsion in an acceptable oil) or ion exchange resins, or as sparingly soluble derivatives; e.g., as a sparingly soluble salt form of the apolipoprotein.

Alternatively, transdermal delivery systems manufactured as an adhesive disc or patch that slowly releases the active ingredient for percutaneous absorption can be used. To this end, permeation enhancers can be used to facilitate transdermal penetration of the active ingredient. A particular benefit may be achieved by incorporating the apolipoprotein, lecithin cholesterol acyltransferase or paroxnase or lipid complexes thereof into a transdermal patch with nitroglycerin for use in patients with ischemic heart disease and hypercholesterolemia.

For oral administration, the pharmaceutical formulations can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art (see, Remington's Pharmaceutical Sciences, 18^{th} Edition Gennaro et al., eds.) Mack Printing Company, Easton, Pa., 1990).

Liquid pharmaceutical compositions for oral administration can take the form of, for example, solutions, syrups or suspensions, or they can be a dry product for constitution with water or other suitable vehicle before use. Such liquid pharmaceutical compositions can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid.).

The pharmaceutical compositions can also comprise buffer salts, flavoring, coloring and sweetening agents as appropriate. Pharmaceutical compositions for oral administration can be suitably prepared to provide controlled release of the apolipoprotein, lecithin cholesterol acyltransferase or paroxnase or lipid complexes thereof.

Enteral pharmaceutical compositions can be suitable for buccal administration, for example, in the form of tablets, troches or lozenges. For rectal and vaginal routes of administration, the apolipoprotein, lecithin cholesterol acyltransferase or paroxnase or lipid complexes thereof can be prepared as solutions (e.g., for retention enemas) suppositories or ointments. Enteral pharmaceutical compositions can be suitable for admixture in feeding mixtures, such as for mixture with total parenteral nutrition (TPN) mixtures or for delivery by a feeding tube (see, Dudrick et al., 1998, Surg. Technol. Int. VII: 174-184; Mohandas et al., 2003, Natl. Med. J India 16(1):29-33; Bueno et al., 2003, Gastrointest. Endosc. 57(4):536-40; Shike et al., 1996, Gastrointest. Endosc. 44(5):536-40).

For administration by inhalation, the apolipoprotein, lecithin cholesterol acyltransferase or paroxnase or lipid complexes thereof can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated comprising a powder mix of the compound and a suitable powder base such as lactose or starch. Inhaled pharmaceutical compositions can be useful, for example, for treating or preventing lung tissue damage during or after heart-lung transplant.

The compositions can, if desired, be presented in a pack or dispenser device that can comprise one or more unit dosage forms comprising the apolipoprotein, lecithin cholesterol acyltransferase or paroxonase or lipid complexes thereof. The pack may for example comprise metal or plastic foil, such as a blister pack. The pack or dispenser device may be accompanied by instructions for administration.

Various embodiments of the pharmaceutical compositions have been described. The descriptions and examples are intended to be illustrative of the invention and not limiting. Indeed, it will be apparent to those of skill in the art that modifications to the pharmaceutical compositions may be made to the various embodiments of the invention described without departing from the spirit of the invention.

5.7. Methods of Treatment

The methods and compositions of the present invention can be used to treat or prevent any condition associated with ischemic reperfusion injury or reduce ischemic reperfusion injury. Ischemic reperfusion injury can be associated with oxygen deprivation, neutrophil activation and myeloperoxidase production. Ischemic reperfusion injury can be the result of a number of disease states or can be iatrogenically induced, for example, blood clots, stenosis or surgery can all cause ischemic reperfusion injury. For purposes of this section and section 5.6, below, a “patient” or “individual” refers to an animal, including a human, in need of treatment, amelioration or reduction of injury from ischemic reperfusion.

In certain embodiments, the methods and compositions of the present invention can be used to treat or prevent conditions associated with oxygen deprivation, neutrophil activation and myeloperoxidase production. In certain embodiments, the methods and compositions can be used to treat, reduce or prevent ischemic reperfusion injury due to blood clots (either one or more than one clot), stenosis, surgery or mechanical obstruction.

In certain embodiments, the methods and compositions of the present invention can be used to treat or prevent stroke, a fatal or non-fatal myocardial infarction, peripheral vascular disease, tissue necrosis, and kidney failure, and post-surgical loss of muscle tone resulting from ischemic reperfusion injury. In certain embodiments, the methods and compositions of the present invention reduce or mitigate the extent of ischemic reperfusion injury. Creatine kinase can be a measure of tissue or organ injury. Thus, in certain embodiments, the methods and compositions of the present invention reduce the amount of tissue or organ creatine kinase.

In certain embodiments, the methods and compositions of the present invention can be used to treat, reduce or prevent ischemic reperfusion injury associated with occlusion or blood diversion due to vessel stenosis, thrombosis, accidental vessel injury, or surgical procedures. Stenosis can be the result of a medical condition such as atherosclerosis or iatrogenically induced, such as a surgical procedure. Surgical procedures, for example, on the knee, hand, hip and shoulder, tissue transplantation and cardiac procedures including coronary artery bypass graft, percutaneous transluminal coronary angioplasty can all reduce or stop blood flow, induce ischemia and set the stage for reperfusion injury. In certain embodiments, the methods and compositions of the present invention can be used to treat, reduce or prevent ischemic reperfusion injury due to stenosis, including atherosclerosis, or surgery, including, but not limited to, surgery on the knee, hand, hip and shoulder, tissue transplantation and cardiac procedures including coronary artery bypass graft, percutaneous transluminal coronary angioplasty. The methods and compositions of the present invention can also be used to treat or prevent any other condition associated with ischemic reperfusion such as myocardial infarction, stroke, intermittent claudication, peripheral arterial disease, acute coronary syndrome, cardiovascular disease and muscle damage as a result of occlusion of a blood vessel.

In certain embodiments, the methods and compositions of the invention can be used to treat, prevent or reduce ischemia reperfusion injury in extracorporeal tissue or organs. Extracorporeal tissue or organs are tissue or organs not in an individual (also termed ex vivo), such as in transplantation. For tissue and organ transplantation, donor tissue and organs removed are also susceptible to reperfusion injury during harvesting, while in transit and following transplantation into a recipient. The methods and compositions can be used to increase the viability of a transplantable tissue or organ by, for example, supplementing solutions used to maintain or preserve transplantable tissues or organs. For example, the methods and compositions can be used to bathe the transplantable tissue or organ during transport or can be placed in contact with the transplantable tissue or organ prior to, during or after transplantation.

In certain embodiments, the methods and compositions can be used to reduce or even to obviate the need for coronary artery bypass surgery in an individual. In other embodiments, the methods and compositions of the invention can be used to treat or prevent conditions associated with percutaneous transluminal coronary angiography, such as percutaneous transluminal coronary angiography induced occlusion. In further embodiments, the methods and compositions of the invention can be used to reduce the recovery time from any surgical procedure. In certain embodiments, the methods and compositions can be pharmaceutically acceptable compositions for pericardial, intracoronary or intraarterial administration during cardiac surgery. In certain embodiments, the pharmaceutically acceptable composition can be administered by a mechanical device such as a pump or perfuser (e.g., perDUCER®).

The methods and compositions can be used in conjunction with cardiac surgery, for example, in or with cardioplegic solutions to prevent or minimize ischemia or reperfusion injury to the myocardium. In certain embodiments, the methods and compositions can be used with a cardiopulmonary bypass machine during cardiac surgery to prevent or reduce ischemic reperfusion injury to the myocardium.

In certain embodiments, the methods and compositions can be practiced as a single, one time dose or chronically. By chronic it is meant that the methods and compositions of the invention are practiced more than once to a given individual. For example, chronic administration can be multiple doses of a pharmaceutical composition administered to an animal, including an individual, on a daily basis, twice daily basis, or more or less frequently, as will be apparent to those of skill in the art. In another embodiment, the methods and compositions are practiced acutely. By acute it is meant that the methods and compositions of the invention are practiced in a time period close to or contemporaneous with the ischemic or occlusive event. For example, acute administration can be a single dose or multiple doses of a pharmaceutical composition administered at the onset of an acute myocardial infarction, upon the early manifestation of, for example, a stroke, or before, during or after a surgical procedure. A time period close to or contemporaneous with an ischemic or occlusive event will vary according to the ischemic event but can be, for example, within about 30 minutes of experiencing the symptoms of a myocardial infarction, stroke or intermittent claudication. In certain embodiments, acute administration is administration within about an hour of the ischemic event. In certain embodiments, acute administration is administration within about 2 hours, about 6 hours, about 10 hours, about 12 hours, about 15 hours or about 24 hours after an ischemic event.

By multiple doses, it is meant that the composition is administered more than once. Multiple doses can be, for example, one dose administered about daily on more than one day, more than one dose administered on one day or multiple doses administered on multiple days.

5.8. Combination Therapy

The apolipoprotein, lecithin cholesterol acyltransferase or paroxonase or lipid complexes thereof or pharmaceutical compositions thereof can be used alone or in combination therapy with other drugs in the methods of the present invention. Such therapies include, but are not limited to simultaneous or sequential administration of the drugs involved.

For example, the apolipoproteins, lecithin cholesterol acyltransferase or paroxonase or lipid complexes thereof or pharmaceutical compositions thereof can be administered with other pharmaceutically active agents including, but not limited to, alpha/beta adrenergic antagonists, antiadrenergic agents, alpha-1 adrenergic antagonists, beta adrenergic antagonists, AMP kinase activators, angiotensin converting enzyme (ACE) inhibitors, angiotensin II receptor antagonists, calcium channel blockers, antiarrhythmic agents, vasodilators, nitrates, vasopressors, inotropic agents, diuretics, anticoagulation agents, antiplatelet aggregation agents, thrombolytic agents, antidiabetic agents, antioxidants, anti-inflammatory agents, bile acid sequestrants, statins, cholesterol ester transfer protein (CETP) inhibitors, cholesterol reducing agents/lipid regulators, drugs that block arachidonic acid conversion, estrogen replacement therapy, fatty acid analogues, fatty acid synthesis inhibitors, fibrates, histidine, nicotinic acid derivatives, peroxisome proliferator activator receptor agonists or antagonists, fatty acid oxidation inhibitors, thalidomide or thiazolidinediones (Drug Facts and Comparisons, updated monthly, January 2003, Wolters Kluwer Company, St. Louis, Mo.; Physicians Desk Reference (56^{th} edition, 2002) Medical Economics). Such agents can have additive or synergistic affects in preventing ischemic-reperfusion injury.

Other agents singly or in combination, that can add to or can synergize the beneficial properties of the apolipoprotein in protecting tissue and organs of a mammal from ischemic reperfusion injury include but are not limited to: Alpha/Beta Adrenergic Antagonists such as, carvediol, (Coreg®); labetalol HCl, (Normodyne®); Antiadrenergic Agents such as guanadrel, (Hylorel®); guanethidine, (Ismelin®); reserpine, clonidine, (Catapres® Catapres-TTS®); guanfacine, (Tenex®); guanabenz, (Wytensin®); methyldopa and methyldopate, (Aldomet®); Alpha-I Adrenergic Antagonist such as doxazosin, (Cardura®); prazosin, (Minipress®); terazosin, (Hytrin®); and phentolamine, (Regitine®); Beta Andrenergic Antagonists such as sotalol, (Betapace AF® and Betapace®); timolol, (Blocadren®); propranolol, (InderalLA® and Inderal®); betaxolol, (Kerlone®); acebutolol, (Sectral®); atenolol, (Tenormin®); metoprolol, (Lopressor® and Toprol-XL®); bisoprolol, (Zebata®); carteolol, (Cartrol®); esmolol, (Brevibloc®); naldolol, (Corgard®); penbutolol, (Levatol®); and pindolol, (Visken®); AMP kinase activators such as ESP 31015, (ETC-1001); ESP 31084, ESP 31085, ESP 15228, ESP 55016 and ESP 24232; gemcabene (PD 72953 and CI-1027); and MEDICA 16; Angiotensin Converting Enzyme (ACE) Inhibitors such as quinapril, (Accupril®); benazepril, (Lotensin®); captopril, (Capoten®); enalapril, (Vasotec®); ramipril, (Altace®); fosinopril (Monopril®); moexipril, (Univasc®); lisinopril, (Prinivl® and Zestril®); trandolapril, (Mavik®), perindopril, (Aceon®); and Angiotension II Receptor Antagonists such as candesaartan, (Atacand®); irbesartan, (Avapro®); losartan, (Cozaar®); valsartan, (Diovan®); telmisartan, (Micardis®); eprosartan, (Tevetan®); and olmesartan, (Benicar®); Calcium Channel Blockers such as nifedipine, (Adalat®, Adalat CC®, Procardia® and Procardia XL®); verapamil, (Calan®, CalanSR®, Covera-HS®, IsoptinSR®, Verelan® and VerelanPM®); diltiazem, (Cardizem®, CardizemCD® and Tiazac®); nimodipine, (Nimotop®); amlodipine, (Norvasc®); felodipine, (Plendil®); nisoldipine, (Sular®); bepridil, (Vascor®); isradipine, (DynaCirc®); and nicardipine, (Cardene®); Antiarrhythmics such as various quinidines; procainamide, (Pronestyl® and Procan®); lidocaine, (Xylocaine®); mexilitine, (Mexitil®); tocainide, (Tonocard®); flecainide, (Tambocor®)); propafenone (Rythmol®), moricizine, (Ethmozine®)); ibutilide, (Covert®); disopyramide, (Norpace®); bretylium, (Bretylol®); amiodarone, (Cordarone®); adenosine, (Adenocard®); dofetilide (Tikosyn®); and digoxin, (Lanoxin®); Vasodilators such as diazoxide, (Hyperstat IV®)); hydralazine, (Apresoline®); fenoldopam, (Corolpam®); minoxidil, (Loniten®); and nitroprusside, (Nipride®); Nitrates such as isosorbide dinitrate; (Isordil® and Sorbitrate®); isosorbide mononitrate, (Imdur®, Ismo® and Monoket®); Nitroglycerin paste, (Nitrol®); various nitroglycerin patches; nitroglycerin SL, (Nitrostat®), Nitrolingual spray; and nitroglycerin IV, (Tridil®); Vassopressors such as norepinephrine, (Levophed®)); and phenylephrine, (Neo-Synephrine®); Inotrophic Agents such as amrinone; (Inocor®); dopamine, (Intropine®); dobutamine, (Dobutrex®); epinephrine, (Adrenalin®); isoprotemol, (Isuprel®), milrinone, (Primacor®); Diuretics such as spironolactone, (Aldactone®); torsemide, (Demadex®); hydroflumethiazide, (Diucardin®); chlorothiazide, (Diuril®); ethacrynic acid, (Edecrin®); hydrochlorothiazide, (hydrochlorothiazide® and Microzide®); amiloride, (Midamor®); chlorthalidone, (Thalitone® and Hygroton®); bumetanide, (Bumex®); furosemide, (Lasix®); indapamide, (Lozol®); metolazone, (Zaroxolyn®); triamterene, (Dyrenium®); and combinations of triamterene and hydrochlorothiazide (Dyazide® and Maxzide®); Anticoagulants/Antiplatelet such as bivalirudin, (Angiomax®); lepirudin, (Refludan®); various heparins; danaparoid, (Orgaran®); various low molecular weight heparins; dalteparin, (Fragmin®); enoxaparin, (Lovenox®); tinzaparin, (Innohep®); warfarin, (Coumadin®); dicumarol, (Dicoumarol®); anisindione, (Miradone®); aspirin; argatroban, (Argatroban®); abciximab, (Reopro®); eptifibatide, (Integrilin®); tirofiban, (Aggrastat®); clopidogrel, (Plavix®); ticlopidine, (Ticlid®); and dipyridamole, (Persantine®); Thrombolytics such as alteplase, (Activase®); tissue plasminogen activator (TPA), (Activase®); anistreplase, APSAC, (Eminase®); reteplase, rPA, (Retavasae®); steptokinase, SK, (Streptase®); urokinase, (Abbokinase®); Antidiabetic agents such as metformin, (Glucophage®); glipizide, (Glucotrol®); chlorpropamide, (Diabinese®); acetohexamide, (Dymelor®); tolazamide, (Tolinase®); glimepride, (Amaryl®); glyburide, (DiaBeta® and Micronase®); acarbose, (Precose®); miglitol, (Glyset®); repaflinide, (Prandin®); nateglinide, (Starlix®); rosiglitazone, (Avandia®); and pioglitazone (Actos®); Antioxidants and anti-inflammatory agents; Bile Acid Sequestrants such as cholestyramine, (LoCholest®, Prevalite® and Questran®); colestipol, (Colestid®); and colesevelam, (Welchol®); Statins such as rovastatin, (Crestor®); fluvastatin, (Lescol®); atorvastatin, (Lipitor®); lovastatin, (Mevacor®); pravastatin, (Pravachol®); and simvastatin, (Zocor®); CETP inhibitors; drugs that block arachidonic acid conversion: Estrogen replacement therapy; Fatty acid analogues such as PD 72953, MEDICA 16, ESP 24232, and ESP 31015; Fatty acid synthesis inhibitors; fatty acid synthesis inhibitors; fatty acid oxidation inhibitors, ranolazine, (Ranexa®); Fibrates such as clofibrate, (Atromid-S®); gemfibrozil, (Lopid®); micronized fenofibrate capsules, (Tricor®); bezafibrate and ciprofibrate; histidine; Nicotinic Acid derivatives such as niacin extended-release tablets, (Niaspan®); Peroxisome proliferator activator receptor agonists and antagonists; thalidomide, (Thalomid®) and compounds described in U.S. Pat. Nos. 6,459,003, 6,506,799 and U.S. Application Publication Nos. 20030022865, 20030018013, 20020077316, and 20030078239 the contents of which are incorporated herein by reference in their entireties.

5.9. Methods of Administration

The apolipoprotein, lecithin cholesterol acyltransferase or paroxnase or lipid complexes thereof can be administered by any suitable route known to those of skill in the art that ensures bioavailability in the circulation. The route of administration can be indicated by the type of pharmaceutical composition, for example, injectable compositions can be administered parenterally, including, but not limited to, intravenous (IV), intramuscular (IM), intradermal, subcutaneous (SC), intracoronary, intraarterially, pericardially, intraarticular and intraperitoneal (IP) injections. In certain embodiments, administration is by a mechanical pump or delivery device, e.g., a pericardial delivery device (PerDUCER®) or cardiopulmonary bypass machine. In certain embodiments, the compositions are administered by injection, via a subcutaneously implantable pump or depot preparation, in amounts that achieve a circulating serum concentration equal to that obtained through parenteral administration, as described above.

The methods of the invention provide for administration of apolipoprotein, lecithin cholesterol acyltransferase or paroxnase or lipid complexes thereof or pharmaceutical compositions thereof through a variety of different treatment regimens. For example, as described above, the methods provide for chronic or single dose administration. The methods provide, for example, for administration acutely (e.g., contemporaneous or closely temporaly related to the ischemic or occlusive event).

In certain embodiments, chronic administration can be several intravenous injections administered periodically during a single day. In another embodiment, chronic administration can be one intravenous injection administered as a bolus or as a continuous infusion daily, about every other day, about every 3 to 15 days, preferably about every 5 to 10 days, and most preferably about every 10 days. Preferably, the dose administered is less than a toxic dose. Preferably during treatment, the dose and dosing schedule will provide sufficient or steady state levels of an effective amount of one or more component of the composition to treat or prevent ischemic reperfusion injury. In certain embodiments, an escalating dose can be administered. In certain embodiments, the composition is administered intermittently. Depending on the needs of the individual, administration can be by slow infusion with a duration of more than about one hour, by rapid infusion of about one hour or less, or by a single bolus injection.

In another embodiment, acute administration can be at the onset of the ischemic or occlusive event or upon manifestation of symptoms of an ischemic or occlusive event. In one embodiment, the methods provide for acute administration of the compositions of the invention, for example, by emergency medical technicians or qualified person (e.g., medically trained firefighters or police) responding to an emergency call for a possible myocardial infarction. In another embodiment, the methods can be practiced acutely, for example, by administering the compositions after the manifestations of stroke.

The actual dose of the compositions of the invention will vary with the route of administration, the height, weight, age and severity of illness of the patient, the presence of concomitant medical conditions and the like. The compositions of the invention will generally be used in an amount effective to achieve the intended purpose. Of course, it is to be understood that the amount used will depend on the particular application.

For example, for use to prevent ischemic reperfusion injury, a prophylactically effective amount of the composition can be applied or administered to an animal or human in need thereof. By prophylactically effective amount is meant an amount of the composition of the invention that inhibits or reduces the symptoms of ischemic reperfusion injury. The actual prophylactically effective amount will depend on a particular application. An ordinarily skilled artisan will be able to determine prophylactically effective amounts of particular compositions for particular applications without undue experimentation using, for example, the in vitro assays and in vivo assays known to those of skill in the art. Exemplary assays are described in the examples below.

For use to treat or prevent diseases related to ischemic reperfusion injury, the compositions of the invention can be administered or applied in a therapeutically effective amount. By therapeutically effective amount is meant an amount effective to ameliorate the symptoms of, or ameliorate, treat or prevent ischemic reperfusion injury. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a beneficial circulating composition concentration range. Initial dosages can also be estimated from in vivo data, e.g., animal models, using techniques that are well known in the art. Such information can be used to more accurately determine useful doses in humans. One having ordinary skill in the art could readily optimize administration to humans based on animal data.

Toxicity of the compositions described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Compositions which exhibit high therapeutic indices are preferred. The data obtained from these cell culture assays and animal studies can be used in formulating a dosage range that is not toxic for use in humans. The dosage of the composition described herein lies preferably within a range of circulating concentrations that include the effective dose with little or no toxicity. The dosage may vary within this range depending upon the dosage form employed and the route of administration utilized. The exact route of administration and dosage of the compositions can be chosen by the individual physician in view of the patient's condition. (See, e.g., Fingl et al., 1975, In: The Pharmacological Basis of Therapeutics, Ch. 1, p. 1).

### 6. EXAMPLES

### 6.1. Example 1

### Ex Vivo Langendorff

This example demonstrates the cardioprotective effect of prophylactic ETC-216 in the reperfused isolated ischemic rabbit heart. Male New Zealand White rabbits, obtained from Charles River weighing approximately 2-3 kg were used in the study. The male New Zealand White rabbit was selected as the appropriate test system for the purposes of this study. The isolated ischemic-reperfused rabbit heart is a model of human myocardial infarction. Upon arrival, animals were assigned unique identification numbers.

Animals were housed in stainless steel cages in accordance with the guidelines of the University of Michigan Committee on the Use and Care of Animals. Veterinary Care provided by the University of Michigan Unit for the Laboratory Animal Medicine. The University of Michigan is accredited by the American Association of Accreditation of Laboratory Animal Health Care, and the animal care use program conforms to the standards in the Guide for the Care and use of Laboratory Animals, DHEW (NIH) Publ. No. 86-23.

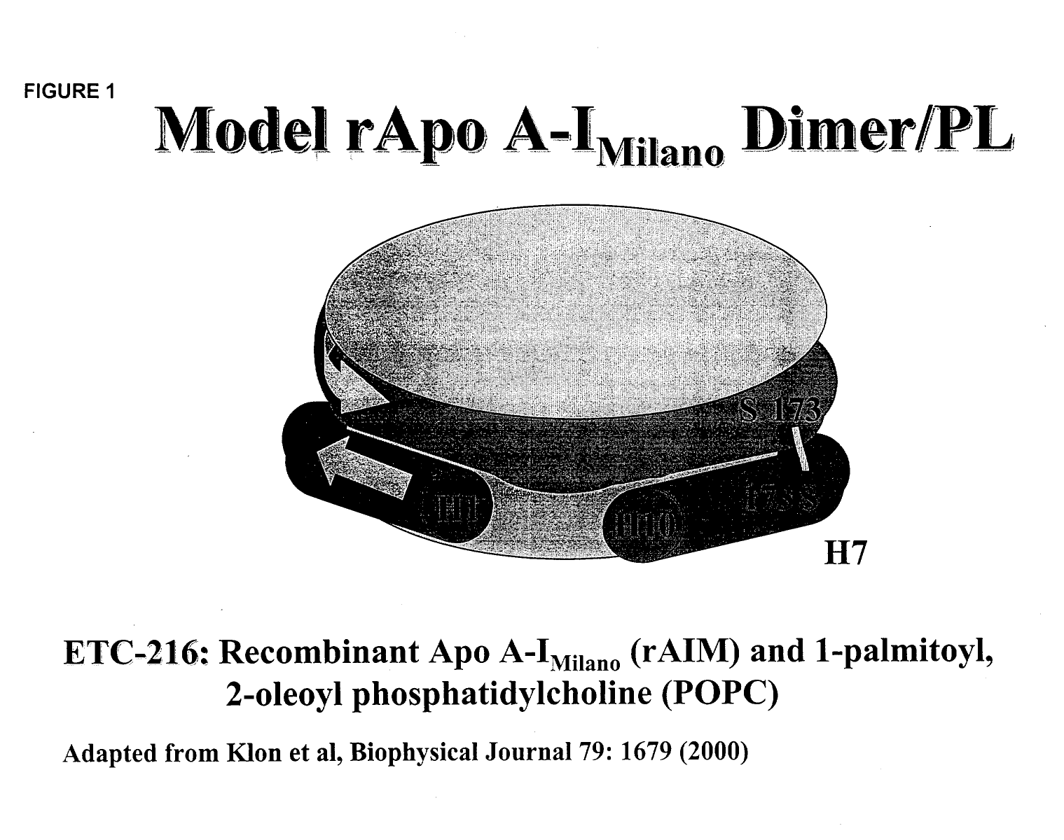

ETC-216 is recombinant apolipoprotein A-I Milano/1-palmitoyl-2-oleoyl phosphatidylcholine complex in a one to one ratio by weight (FIG. 1). Stock solutions of ETC-216 contained 14 mg protein/ml in a sucrose mannitol buffer. Since the sucrose-mannitol buffer was incompatible with Krebs-Henseleit buffer, and to control for any independent effects of mannitol alone, ETC-216 was dialyzed to obtain a background buffer comprised of 2% glucose in 4 mM sodium phosphate, pH 7.4. The ETC-216 was diluted with Krebs-Henseleit buffer to yield a drug concentration of 0.45 mg/ml. The vehicle was similarly diluted.

Dose selection was based on historical data for doses used in Esperion's Human Phase I single dose safety clinical trials, where doses up to 100 mg/kg of ETC-216 were administered to humans. For the studies outlined in this protocol a concentration of 0.5 mg/ml is approximately equivalent to an intravenous dose of 25 mg/kg administered to a human.

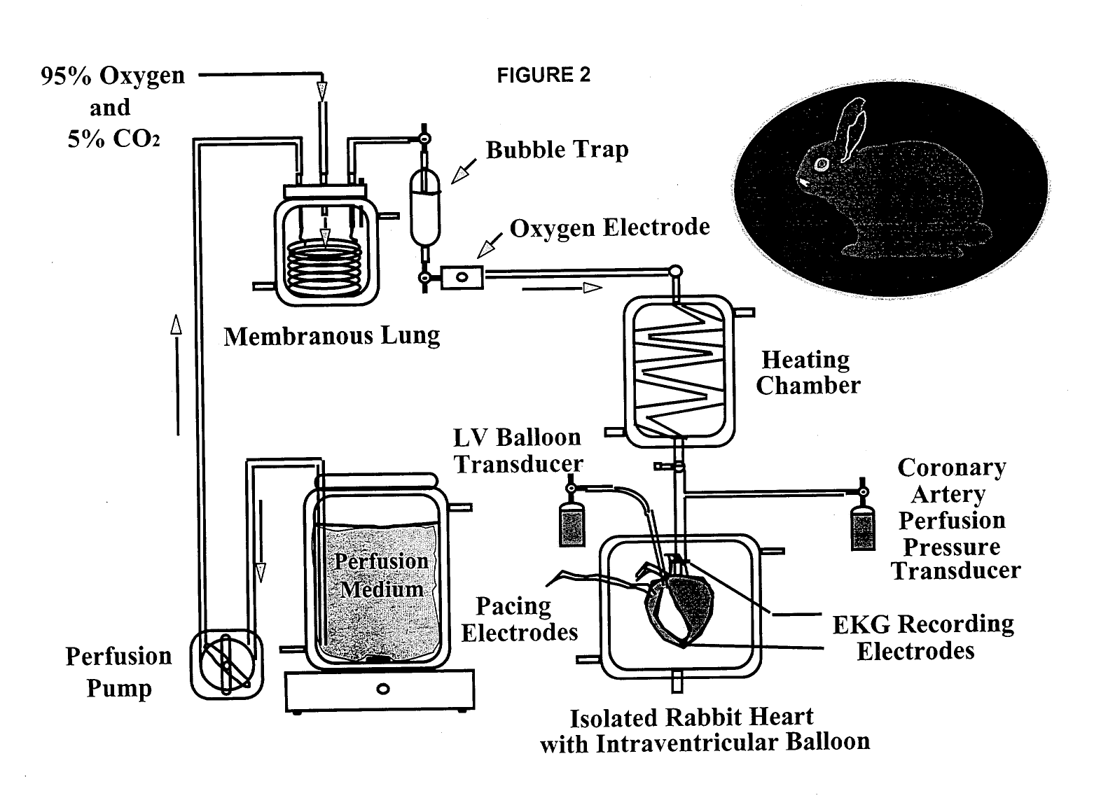

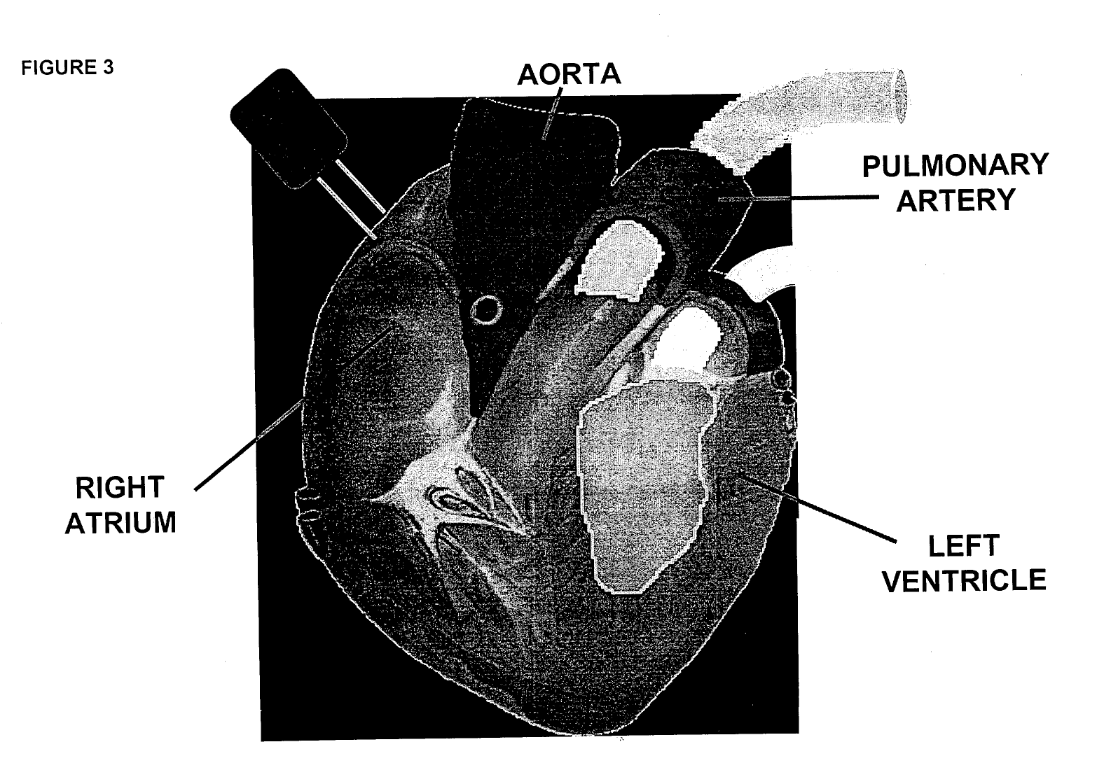

Experiments were conducted using a Langendorff apparatus (FIGS. 2 and 3) to perfuse rabbit hearts. Rabbits were rendered unconscious by cervical dislocation and the hearts were removed rapidly and attached to a cannula for perfusion through the aorta. The perfusion medium consisted of a circulating Krebs-Henseleit buffer (pH 7.4, 37° C.; “KH”) that was exposed continuously to a mixture of 95% O₂/5% CO₂ and delivered at a constant rate of 20-25 ml/min. The hearts were paced throughout the protocol via electrodes attached to the right atrium. Pacing stimuli were delivered from a laboratory square wave generator (10% above physiologic pacing, 1 msec duration, Grass 588, Quincy, Mass.). The pulmonary artery was cannulated with Silastic tubing to facilitate collection of the pulmonary artery effluent representing the coronary venous return to the coronary sinus. The superior and inferior vena cava and the pulmonary veins were ligated to prevent loss of perfusate from the otherwise severed vessels. A left ventricular drain, thermistor probe, and latex balloon were inserted via the left atrium and positioned in the left ventricle. The fluid filled latex balloon was connected with rigid tubing to a Miller Catheter pressure transducer to permit for measurement of left ventricular developed pressure. The intraventricular balloon is expanded with distilled water to achieve an initial baseline left ventricular end-diastolic pressure of approximately 10 mm Hg. Coronary perfusion pressure was measured with a pressure transducer connected to a side-arm of the aortic cannula. Since the rate of coronary artery perfusion was maintained constant, alterations in the coronary artery perfusion pressure served as an indicator of change in coronary artery resistance. All hemodynamic variables were monitored continuously using a multichannel recorder such as a Grass Polygraph 79D (Quincy, Mass.) interfaced to a Polyview Software Data Acquisition System. Hearts were maintained at 37° C. throughout the experimental period by enclosing the heart in a temperature regulated double lumen glass chamber and passing the perfusion medium through a heated reservoir and delivery system.

Two Treatment groups were used for the Experimental Procedures as shown below.

| Group | Treatment | Test Substance | Conc (mg/ml) |
|---|---|---|---|
| 1 | Ischemia & Reperfusion | Vehicle | 0 |
| 2 | Ischemia & Reperfusion | ETC-216 | 0.45 |

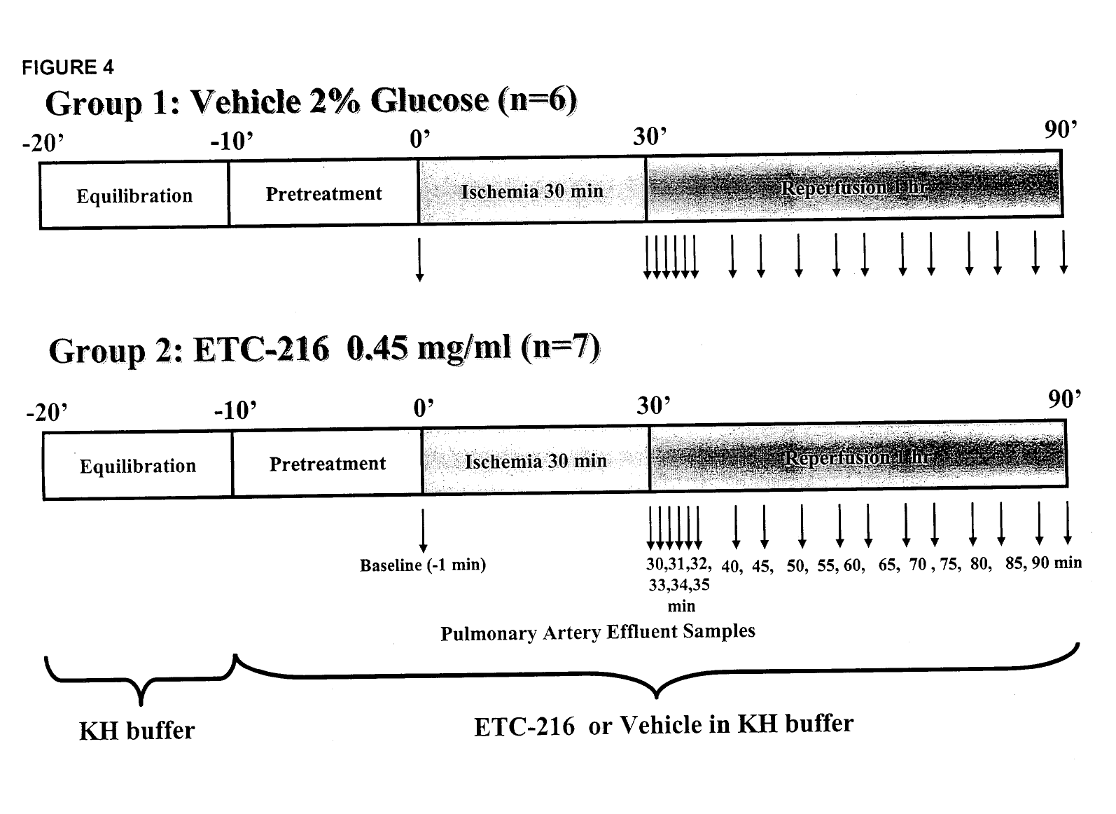

The hearts were experimentally treated as shown in FIG. 4. Isolated hearts were stabilized under normoxic (normal level of oxygen) conditions for 20 minutes before the induction of global ischemia. During the first 10 minutes of this period hearts were exposed to the KH buffer alone, and then for an additional 10 minutes to the KH buffer containing either vehicle (Group 1) or ETC-216 (Group 2). The hearts were then subject to a 30 minute period of ischemia followed by a 60 minute period of reperfusion with KH buffer containing vehicle (Group 1) or ETC-216 (Group 2). Induction of total global ischemia was accomplished by stopping the flow of perfusate to the heart, and reperfusion of the heart was accomplished by turning on the pump to restore the original flow rate.

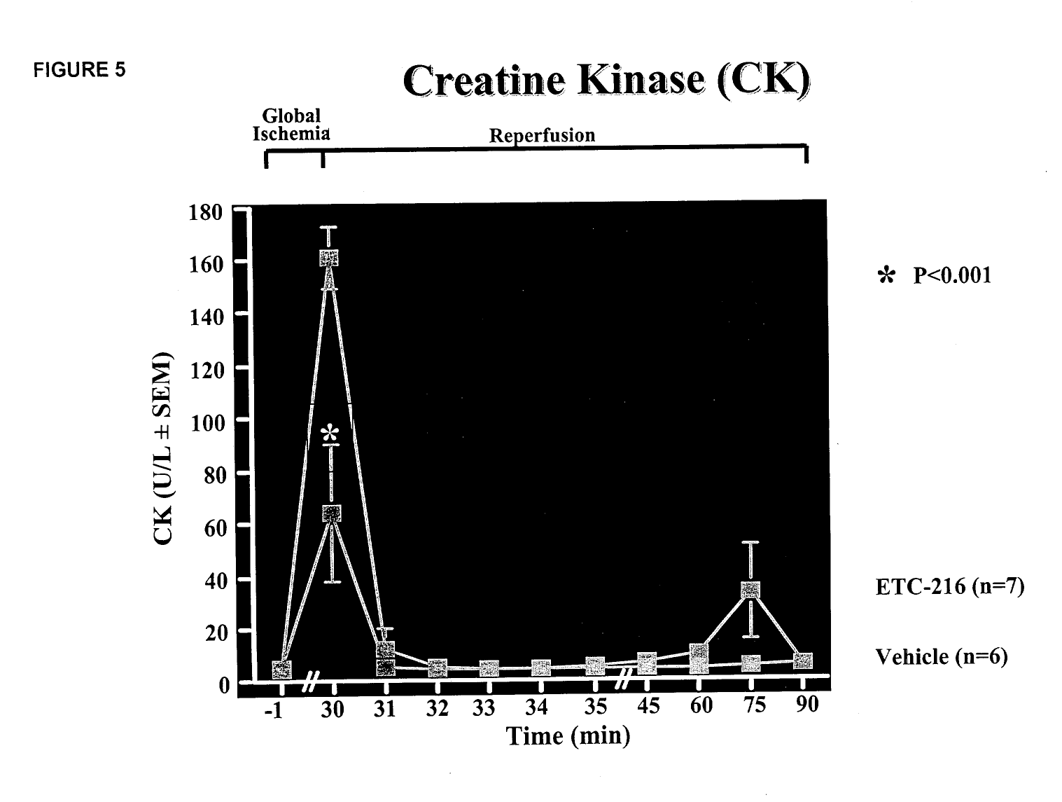

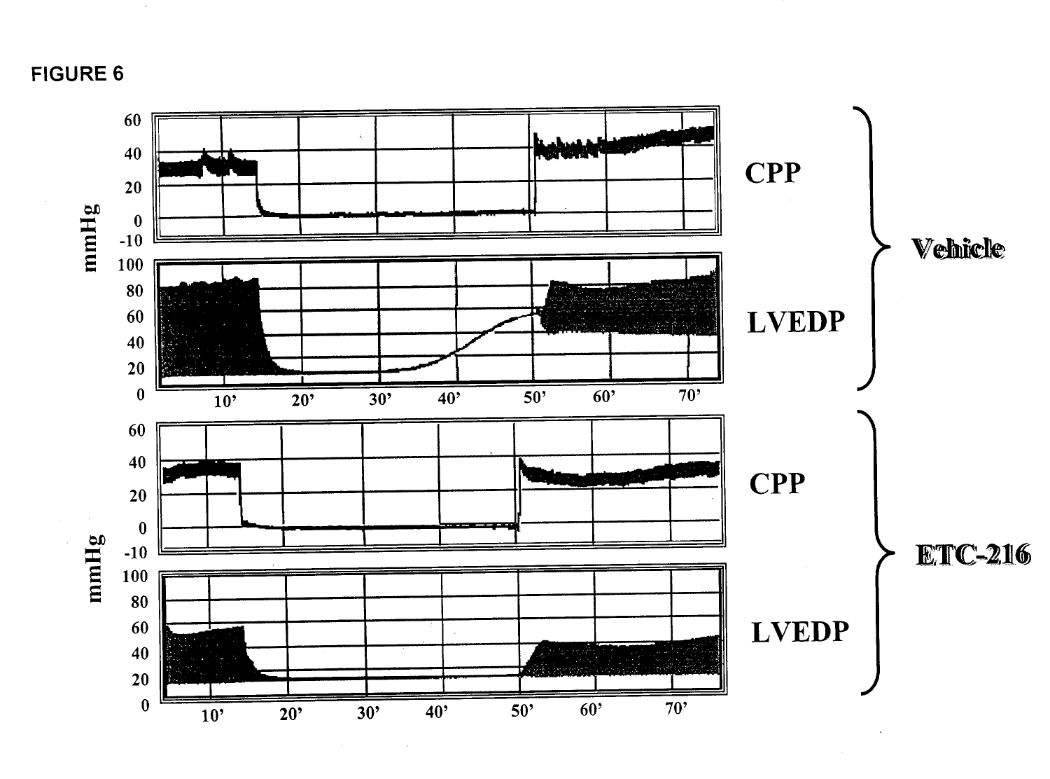

Aliquots of the pulmonary artery effluent were collected from hearts in all groups at baseline (pre-ischemia), and initially every minute up to 5 minutes, and every 5 minutes thereafter during the reperfusion period. The effluents were analyzed for creatine kinase concentration (FIG. 5), an index of tissue injury. Creatine kinase is a cytosolic enzyme released from irreversibly injured cells. Cardiac functions were continuously monitored (FIG. 6).

Heart end-point determinations were made for:

1-Electrocardiogram-heart rate (paced) to detect for the presence or absence of arrhythmias;

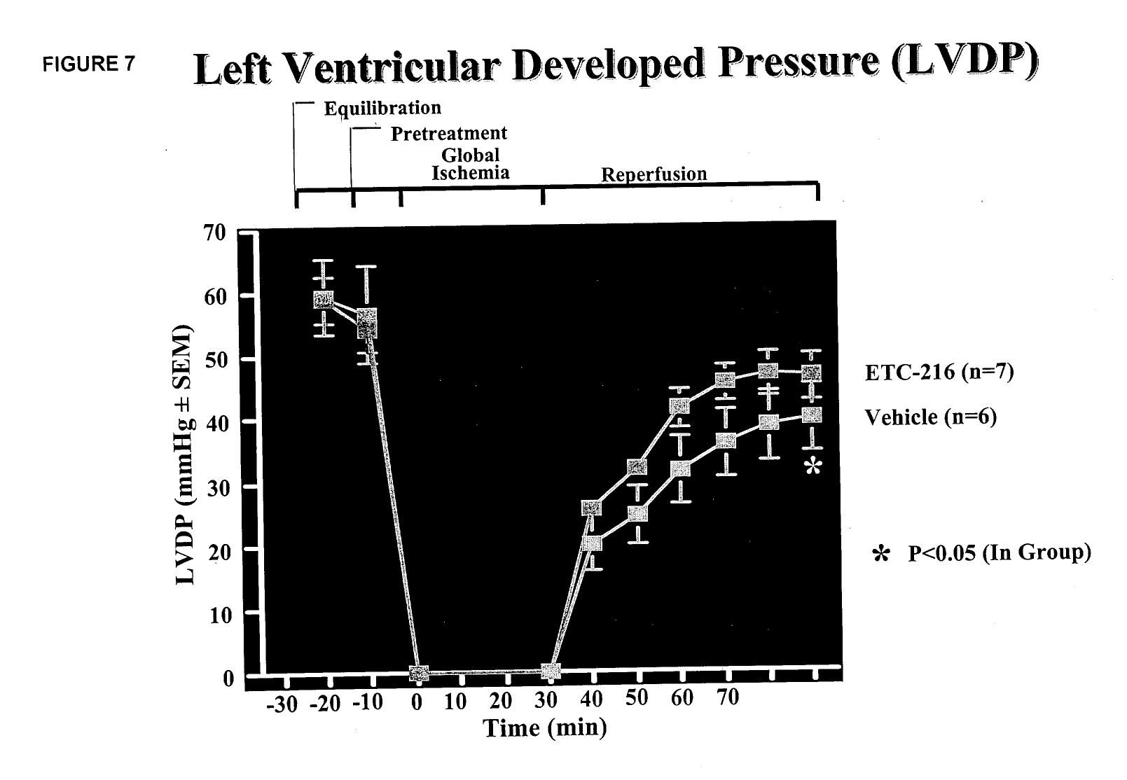

2-Left ventricular developed pressure (FIG. 7) (data shown as mean±standard error of the mean for the indicated number of hearts in each group);

3-Left ventricular dP/dt

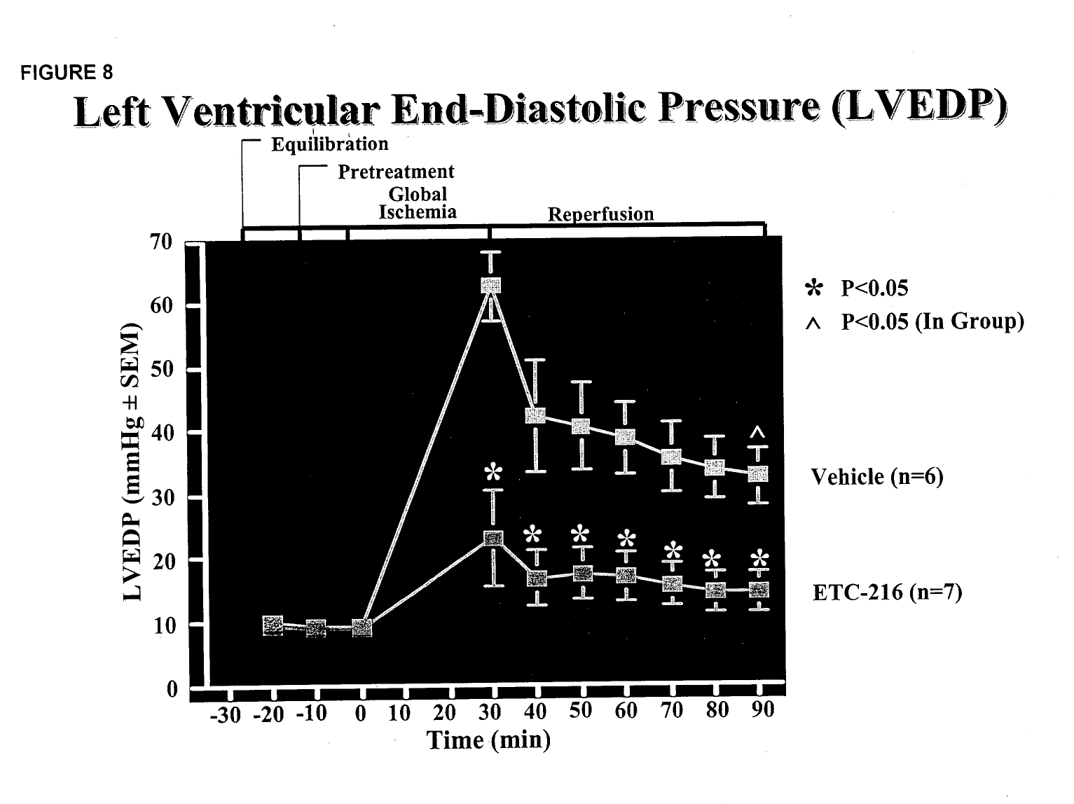

4-Left ventricular end-diastolic pressure (FIG. 8) (data shown as mean±standard error of the mean for the indicated number of hearts in each group);

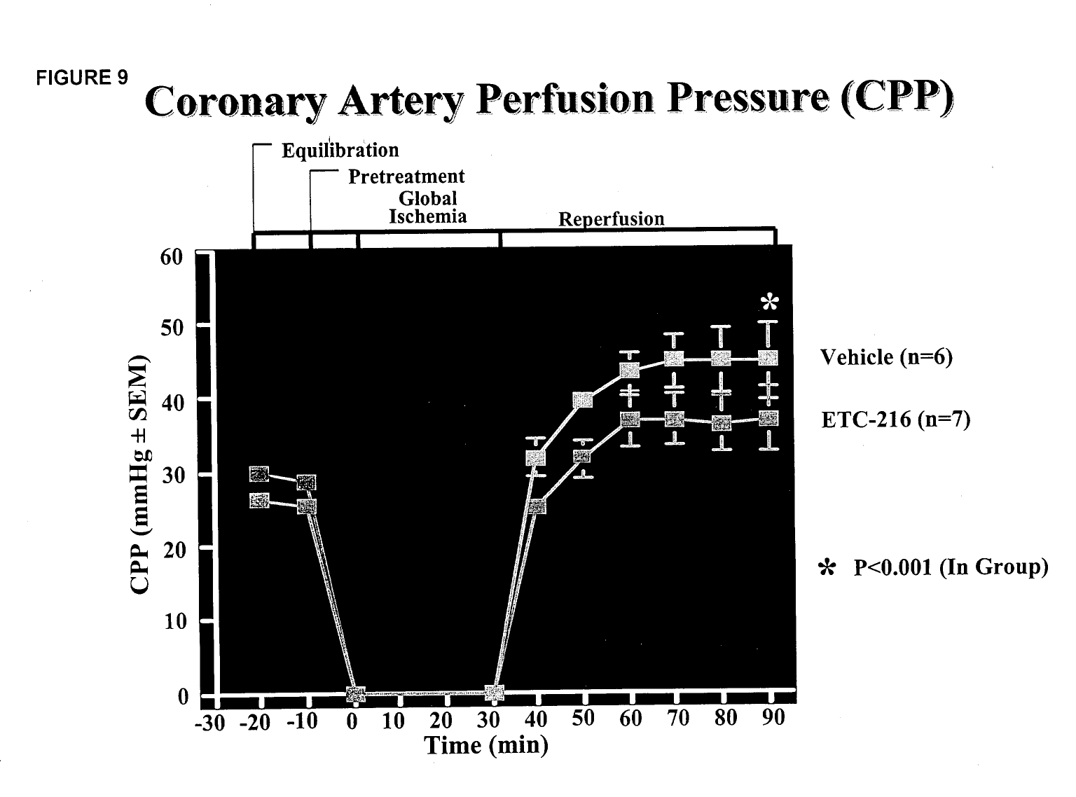

5-Coronary perfusion pressure (FIG. 9) (data shown as mean±standard error of the mean for the indicated number of hearts in each group);

6-Collection of lymphatic drainage to determine release of tissue creatine kinase before and after reperfusion (FIG. 5)

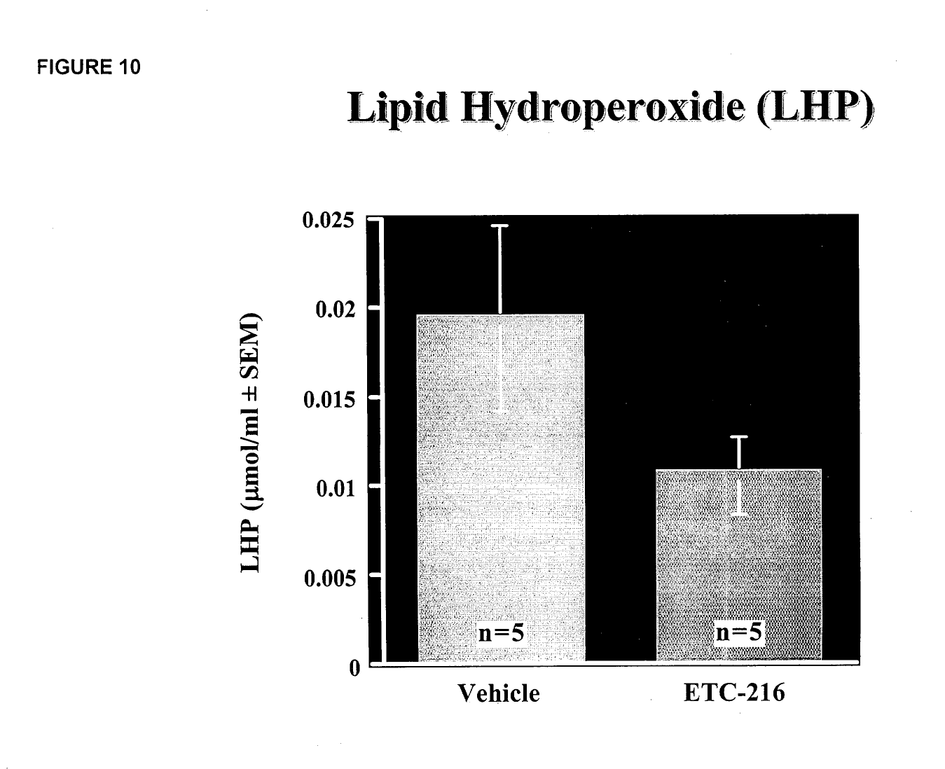

At the conclusion of the experimental protocol, heart biopsies from up to five hearts from each treatment group were immersed immediately in liquid nitrogen and stored at −80° C. for subsequent lipid hydroperoxides analysis. The homogenate samples were normalized to protein content before conducting an assay for lipid peroxides (FIG. 10). ETC-216 reduced cardiac lipid hydroperoxides by 46% in this example.

Upon completion of the designated protocol, two hearts from each group were perfused for 3 minutes with 2.5% glutaraldehyde and 1% LaCl₃ in 0.1 M sodium calcodylate buffer (pH 7.4). The osmophilic LaCl₃ under normal conditions is retained in the vascular compartment bound to the vessel wall and serves as an indicator of blood vessel integrity. Extravasation of LaCl₃ into the extravascular space was used to indicate the presence of vascular injury. Tissue samples from the left ventricular myocardium were removed and cut into segments measuring approximately 1 mm on a side. The samples were fixed for an additional 2 hours at 4° C. in the above mentioned buffer. Thereafter, the samples were dehydrated in an ethanol series and embedded in EM bed-812 (Electron Microscopy Sciences, Ft, Washington, Pa.). Tissue blocs were sectioned with a Reichert ultramicrotome and placed onto formvar-coated copper grids followed by staining with 4% uranyl acetate. Sections were observed with a Phillips CM-10 electron microscope.

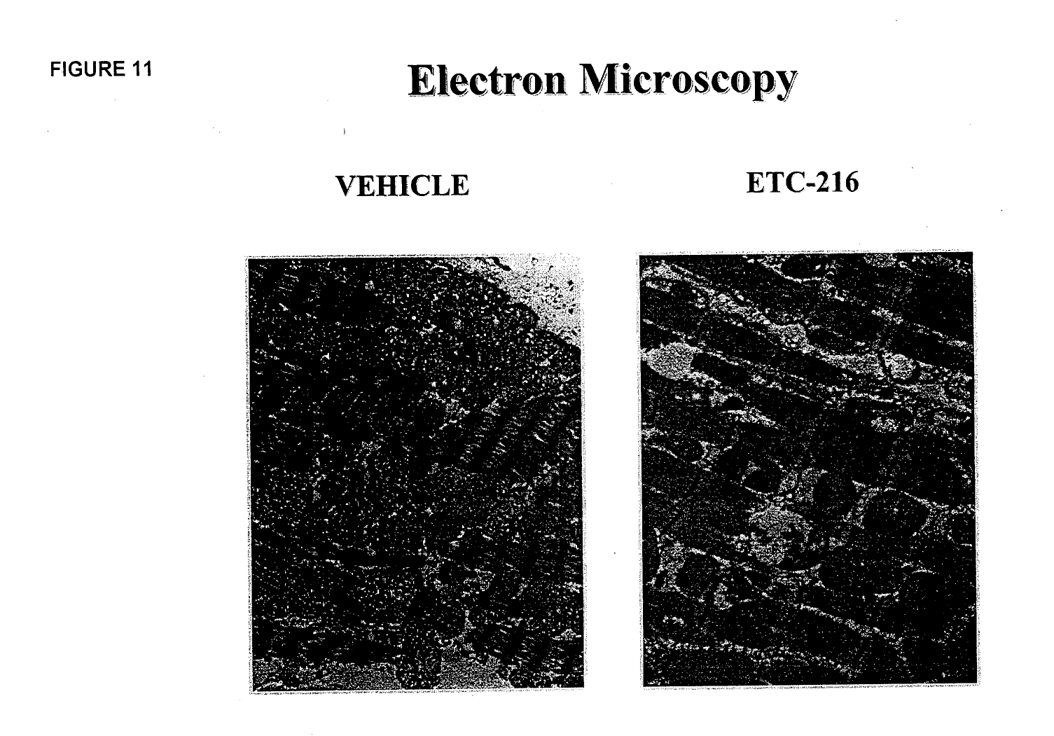

Transmission electron microscopy was used to examine myocardial specimens from each of the study groups. The images show that the vehicle-treated hearts' sarcomere structural features are obliterated and contracture bands are present. The mitochondria are markedly swollen with disrupted crystal and osmophilic inclusion bodies. In the ETC-216 treated hearts, the sarcomere structure is relatively normal and the mitochondria appear intact with only minimal swelling. The virtual absence of contraction bands stands in marked contrast with those observed in the control hearts. The ability of ETC-216 to prevent contraction band necrosis is consistent with the observation that hearts pretreated with ETC-216 did not exhibit an increase in LVEDP upon reperfusion. Both contraction band necrosis and a sustained increase in LVEDP are associated with an increase in intracellular calcium overload and irreversible cell injury. The presence of myofibril blurring of the Z-bands, and disruption of the myofibrillar architecture are indicative of extensive damage. Other expected morphological damage included disrupted cristae and matrices of the mitochondria as well as large, electron dense bodies in the mitochondria. The magnification factor was 7900×in both micrographs (FIG. 11).

Analysis of the creatine kinase concentrations (FIG. 5) indicated that the rapid phase of enzyme release into the venous effluent occurs at the time of reperfusion. Control hearts (treated with vehicle) showed a marked release of creatine kinase compared to the ETC-216 treated hearts. In addition, ETC-216 treated hearts showed reduced left ventricular end-diastolic pressure (FIGS. 6 and 8), elevated left ventricular developed pressure (FIG. 7), decreased coronary artery perfusion pressure (FIG. 9) and decreased lipid hydroperoxide (LHP) compared to control hearts. In addition, ETC-216 protected against morphological changes in the myocardium. These results demonstrate the cardioprotective effects of ETC-216 when administered prior to the ischemic event.

### 6.2. Example 2

### Acute and Chronic Administration in the LAD Occluded-Reperfused Rabbit Heart at 100 mg/kg

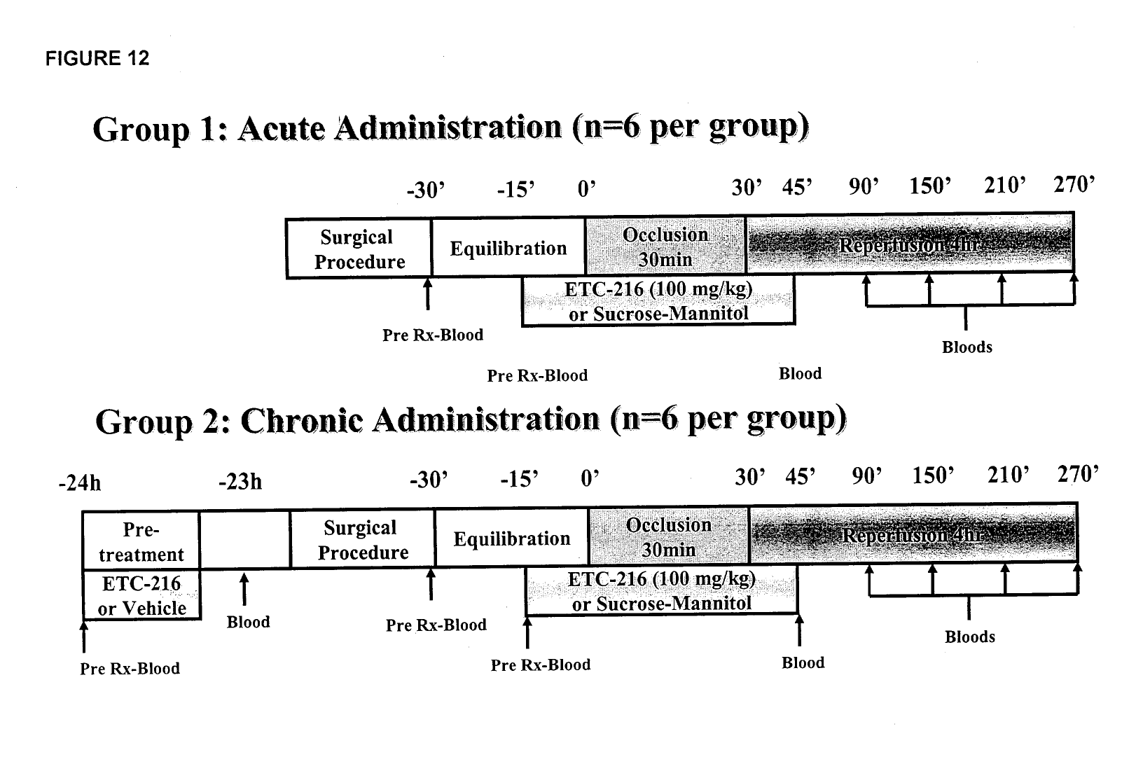

This example demonstrates the cardioprotective effects of ETC-216 in an in vivo model of regional myocardial ischemia and reperfusion. The male New Zealand White rabbit was selected as the appropriate test system for the purposes of this study because of its lack of collateral blood supply to the heart thus making it unnecessary to employ myocardial blood flow determinations. In this study, different dosing regimens were used in separate groups of rabbits that were subjected to 30 minutes of regional myocardial ischemia by coronary artery ligation and reperfusion. Two dosing regimens were used. In the first protocol, ETC-216 was tested as a single pretreatment in which the heart is exposed to 100 mg/kg of the agent just prior to the onset of regional ischemia, while in the second protocol, two 100 mg/kg pretreatments were administered (one day prior and immediately prior) to the onset of regional ischemia. These protocols are shown in (FIG. 12). This study focused on the effects of ETC-216 as a cardioprotective agent in an in vivo study in which the rabbit heart was subjected to regional myocardial ischemia for a period of 30 minutes followed by reperfusion for a minimum of four hours. This example demonstrates that ETC-216 is a cardioprotective agent when given after the ischemic event.

The procedures used in this study are in agreement with the guidelines of the University of Michigan Committee on the Use and Care of Animals. Veterinary care was provided by the University of Michigan Unit for Laboratory Animal Medicine. The University of Michigan is accredited by the American Association of Accreditation of Laboratory Animal Health Care, and the animal care use program conforms to the standards in the Guide for the Care and use of Laboratory Animals DHEW (NIH) Publ. No. 86-23.

Male New Zealand White rabbits obtained from Charles River weighing approximately 2-3 kg were used in the study. Upon arrival, animals were assigned unique identification numbers. Rabbits were anesthetized with a mixture of xylazine (3.0 mg/kg) and ketamine (35 mg/kg) intramuscularly followed by an intravenous injection of sodium pentobarbital (30 mg/kg). Anesthesia was maintained with intravenous injections of a pentobarbital solution (30 mg/ml). A cuffed endotracheal tube was inserted, and animals were placed on positive-pressure ventilation with room air. The right jugular vein was isolated and cannulated for administration of ETC-216 or a matched volume of vehicle. The right carotid artery was isolated, and instrumented with a Millar catheter micro-tip pressure transducer positioned immediately above the aortic valves to monitor aortic blood pressure and to obtain the derived first derivative of the pressure pulse (dP/dt). A lead II electrocardiogram was monitored throughout the experiment. A left thoracotomy and pericardiotomy were performed, followed by identification of the left anterior descending (LAD) coronary artery. A silk suture (3.0; Deknatel, Fall River, Mass.) was passed behind the artery and both ends of the suture were inserted into a short length of polyethylene tubing. Downward pressure on the polyethylene tube while exerting upward tension on the free ends of the suture compresses the underlying coronary artery resulting in occlusion of the vessel and regional myocardial ischemia. The occlusion was maintained for 30 minutes after which the tension on the suture was released and the polyethylene tubing was withdrawn allowing reperfusion to occur. Regional myocardial ischemia was verified by the presence of a region in the area of distribution of the occluded vessel and by changes in the electrocardiogram consistent with the presence of transmural regional myocardial ischemia (ST-segment elevation).

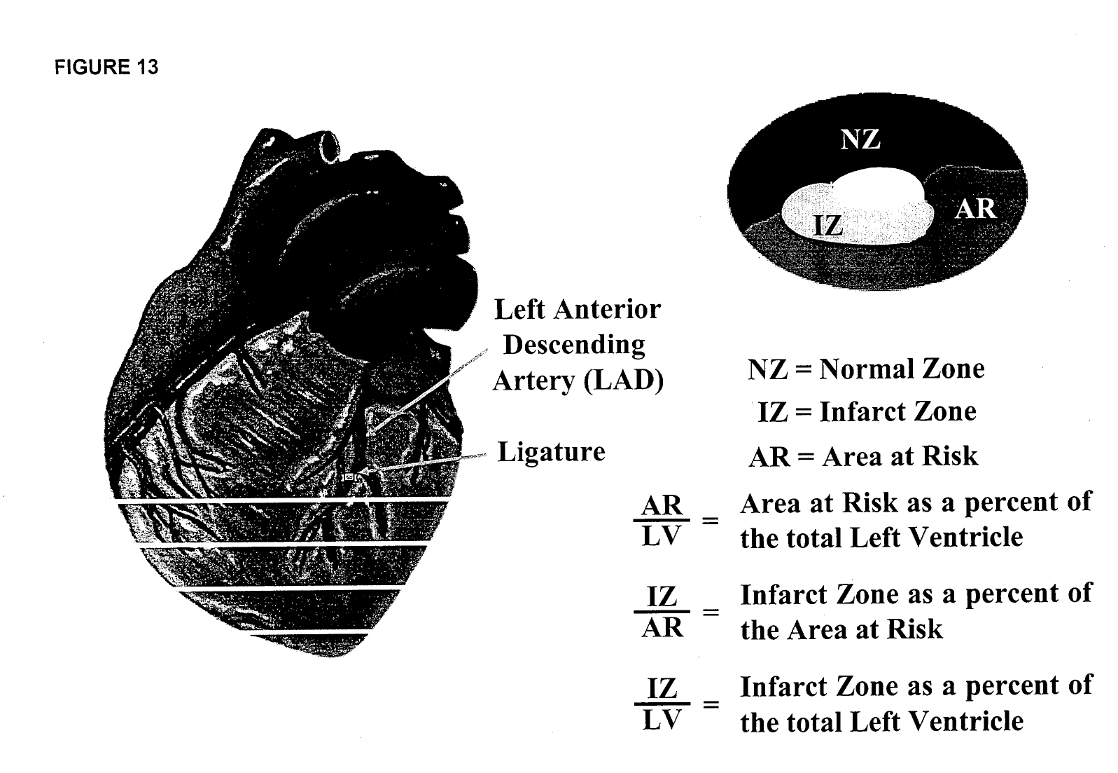

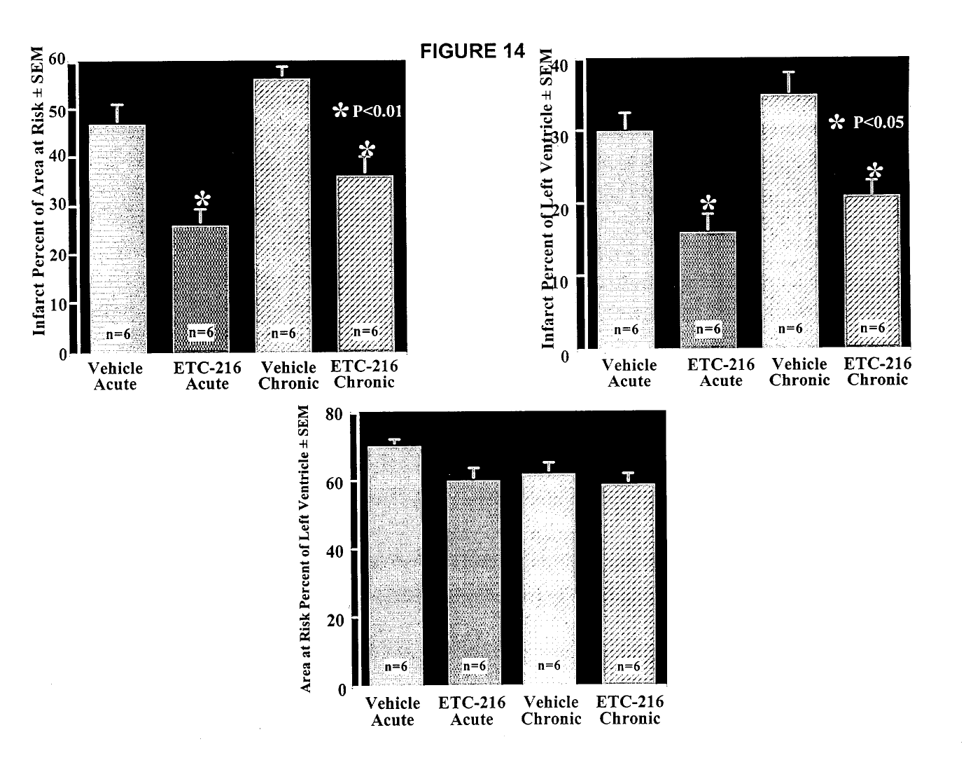

The major end-point determination consisted of measurements of infarct size as a percent of left ventricle and as a percent of the area at risk (FIGS. 13 and 14). At the conclusion of the study, the rabbits, while anesthetized, were given heparin (1,000U intravenously) after which they were euthanized. The heart was excised, and then prepared to be perfused via the aorta on a Langendorff apparatus with Krebs-Henseleit Buffer at a constant flow of 22-24 ml/min. The hearts were washed with buffer for 10 minutes to ensure that the tissue was clean. Forty-five milliliters of a 1% solution of triphenyltetrazolium chloride (TTC) in phosphate buffer (pH 7.4) was perfused through the heart. TTC demarcates the non-infarcted myocardium within the area at risk with a brick-red color, indicating the presence of formazan precipitate resulting from reduction of TTC by coenzymes present in viable myocardial tissue. Irreversibly injured tissue, lacking the cytosolic dehydrogenases, is unable to form the formazan precipitate and appears pale yellow. The left anterior descending (LAD) artery was ligated in a location identical to the area ligated during the induction of regional myocardial ischemia. The perfusion pump was stopped and 2 ml of a 0.25% solution of Evans Blue was injected slowly through a side-arm port connected to the aortic cannula. The dye was passed through the heart for 10 seconds to ensure equal distribution of the dye. Presence of Evans Blue was used to demarcate the left ventricular tissue that was not subjected to regional ischemia, as opposed to the risk region. The heart was removed from the perfusion apparatus and cut into transverse sections at right angles to the vertical axis. The right ventricle, apex, and atrial tissue were discarded. Both surfaces of each transverse section were traced onto clear acetate sheets. The images were photocopied and enlarged. The photocopies were scanned and downloaded into Adobe PhotoShop (Adobe Systems Inc., Seattle, Wash.). The areas of the normal left ventricle (NLV) non-risk regions, area at risk, and infarct are determined by calculating the number of pixels occupying each area using the Adobe Photo Shop Software. Total area at risk is expressed as the percentage of the left ventricle. Infarct size is expressed as the percentage of the area at risk (ARR) (FIGS. 13 and 14).

The infarct percent of area at risk, infarct percent of left ventricle, and area at risk percent of left ventricle in rabbits treated once (i.e., acute treatment) or treated twice (i.e., chronic treatment) with ETC-216 (100 mg/kg) or an equivalent volume of vehicle. Data are mean±standard error of the mean for n=6 animals per group. Asterisks in FIG. 14 indicate significant difference from the respective control.

Other end-point determinations were made. The ultimate infarct size may be influenced by increases or decreases in myocardial oxygen utilization. Two important determinants of myocardial oxygen compensation are heart rate and pressure load. The rate pressure product (heart rate x mean arterial blood pressure) provides an approximation of a change in myocardial oxygen requirements by the heart. Therefore, the rate-pressure product was calculated to determine if an observed reduction in infarct size correlated with the change in the rate pressure product. The heart rate and mean aortic pressure was monitored continuously throughout the experimental protocol and the data was used to calculate the rate pressure product at specific time points in the study for each of the experimental groups.

The area at risk percent of left ventricle was decreased in ETC-216 treated hearts as compared to controls for both acute and chronic administration, however the results were not statistically significant. The infarct percent of area at risk and the infarct percent of left ventricle were significantly decreased in ETC-216 treated hearts as compared to controls for both acute and chronic administration. These results indicate that ETC-216 is cardioprotective when administered both acutely and chronically.

The creatine kinase activity of myocardial tissue in the risk and non-risk regions can be compared. The principle of the assay is based upon an increase in the absorbance of the reaction mixture at 340 nm as a result of the equimolar reduction of NAD to NADH. The rate of change in absorbance is directly proportional to the creatine kinase activity. One unit is defined as the amount of enzyme that produces one micromole of NADPH per minute under the conditions of the assay procedure.

Myocardial tissue subjected to a prolonged period of blood flow deprivation (ischemia) without reperfusion will undergo morphological changes characteristic of necrosis along with the presence of inflammatory cells. The morphologic appearance of ischemia-induced cell death differs from that occurring as a result of reperfusion. The latter is characterized by contraction bands and is referred to as contraction band necrosis. Heart tissue from each of the groups was preserved and prepared for examination by electron microscopy.

Ischemic reperfusion injury is associated with the accumulation of inflammatory cells, predominantly neutrophils, in the area at risk. Myeloperoxidase (MPO) is an enzyme present almost exclusively in neutrophils (Liu et al., J. Pharmacol. Exp. Ther. 287:527-537, 1998). Therefore, it is anticipated that tissue from the respective regions of the heart can be assayed for MPO activity as an indicator of injury. It is also anticipated that an intervention capable of reducing the inflammatory response would be associated with a reduction in MPO activity in the reperfused risk region when compared to heart tissue from the risk region of non-treated animals. Thus, the percent change in MPO activity (risk region/non-risk region) would be reduced in the drug-treated hearts compared to the control vehicle treated hearts.

At the end of the experiment, tissue myeloperoxidase (MPO) activity was determined in a preliminary, uncontrolled, non-validated assay. Heart tissue samples were obtained from the risk region and the non-risk region and were homogenized in 0.5% hexadecyltrimethyl ammonium bromide and dissolved in 50 mM potassium phosphate buffer, pH 6.0 (see also Liu et al., 1998, J. Pharmacol. Exp. Ther. 287:527-537). Homogenates were centrifuged at 12,500 g at 4° C. for 30 minutes. The supernatants were collected and reacted with 0.167 mg/ml o-dianisidine dihydrochloride (Sigma) and 0.0005 percent H₂O₂ in 50 mM potassium phosphate buffer, pH 6.0. The change in absorbance was measured spectrophotometrically at 460 nm. One unit of MPO was defined as that quantity of enzyme hydrolyzing 1 mmol of H₂O₂/minute at 25° C. The results from this preliminary experiment, not presented herein, appear to indicate that there were no differences between ETC-216 and vehicle treated hearts in terms of ischemic reperfusion injury, however, the results have yet to be validated, for example, by comparison of MPO levels prior to ischemic reperfusion injury.

As demonstrated by decreased infarct percent of area at risk and infarct percent of left ventricle, ETC-216 treated hearts were protected from ischemic reperfusion injury. Cardioprotection was conferred by both dosing protocols, that is, ETC-216 administered as a single 100 mg/kg dose prior to the onset of ischemia or ETC-216 administered in two 100 mg/kg doses, one dose given one day prior to ischemia and a second dose given immediately prior to ischemia.

### 6.3. Example 3

### Determination of the Minimal Effective Dose for Acute Administration in the LAD Occluded-Reperfused Rabbit Heart

This example demonstrates the prophylactic efficacy of various doses of ETC-216 when administered as a single pretreatment just prior to the onset of regional ischemia. The study in example 2 focused on the effects of ETC-216 as a cardioprotective agent in an in vivo study in which the rabbit heart was subjected to regional myocardial ischemia for a period of 30 minutes followed by reperfusion for a minimum of four hours. Two dosing regimens were used. In the first protocol, ETC-216 was tested as a single pretreatment in which the systemic circulation was exposed to 100 mg/kg of the agent just prior to the onset of regional ischemia, while in the second protocol, two 100 mg/kg pretreatments were administered prior to (one day prior and immediately prior) to the onset of regional ischemia. Both regimens showed that either one or two treatments with 100 mg/kg ETC-216 is cardioprotective.

Therefore, ETC-216 was tested as a single pretreatment in which the heart was exposed to single doses of the agent or an equivalent volume of vehicle just prior to the onset of regional ischemia to determine effects on cardioprotection. The hearts were analyzed by the same methods used in example 2. In addition, this protocol was designed to find a minimal effect dose of ETC-216 to treat the rabbit heart for protection from ischemia.

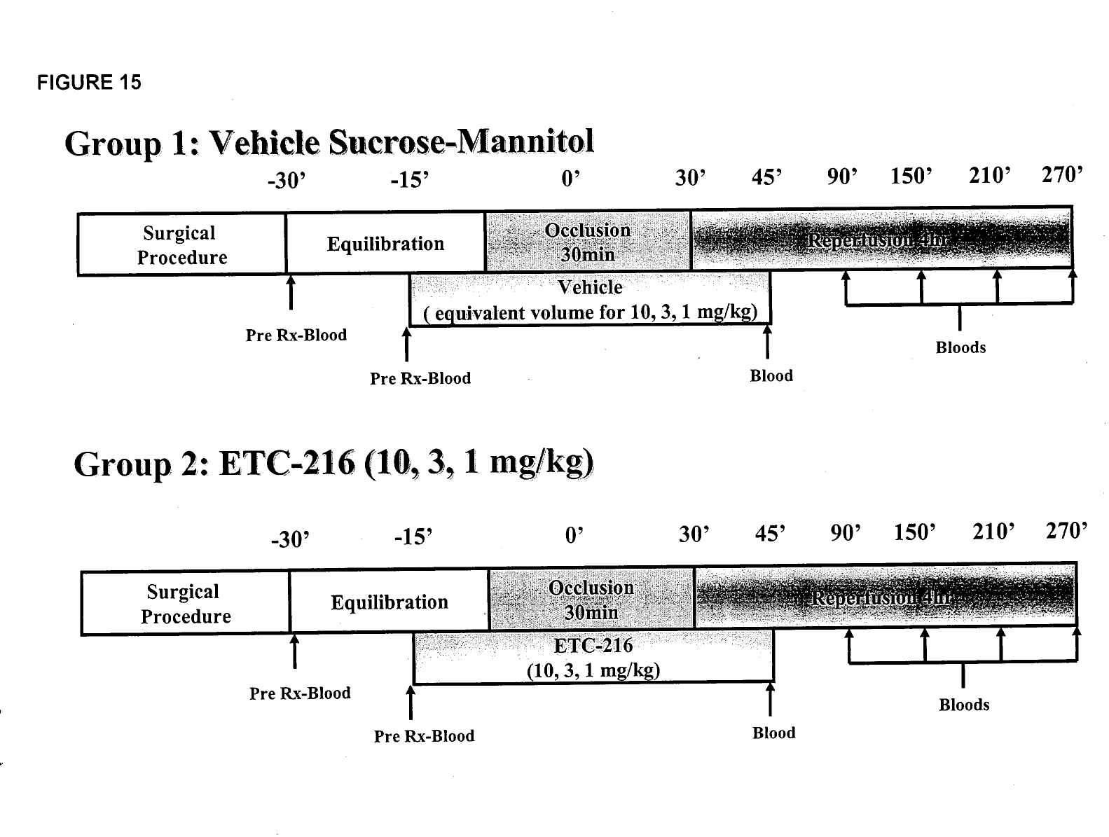

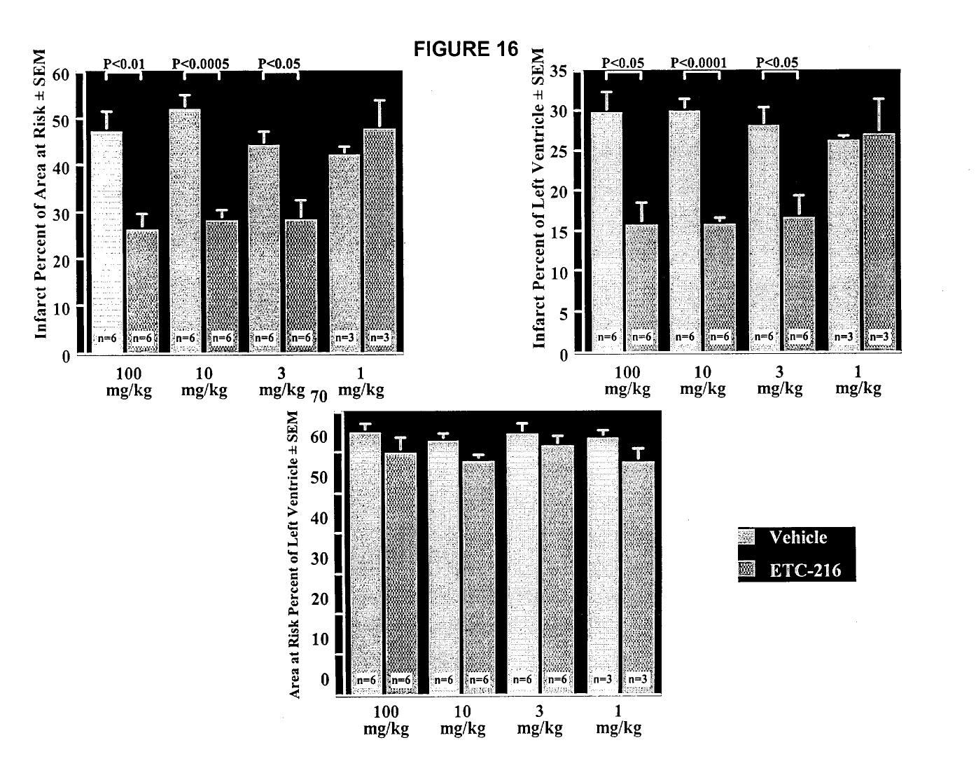

To find the minimal effective dose of ETC-216, the same protocol for the acute treatment (See, FIG. 12) was used in which the animals received single treatments of either 10, 3 or 1 mg/kg of ETC-216 or an equivalent volume of vehicle as shown in FIG. 15. The area at risk (AAR) or ischemic region expressed as a percent of the total left ventricle for the 10 mg/kg treatment group was similar in the control group and in the treatment group (FIG. 16). Rabbits treated with 10 mg/kg ETC-216 developed smaller infarcts (p<0.0005) expressed as a percent of the AAR compared to rabbits treated with vehicle (FIG. 16). A reduction in myocardial infarct size (p<0.0001) was also observed when the data were expressed as a percent of the total left ventricle (FIG. 16).

Similar results were observed with a dose of 3 mg/kg. The AAR expressed as a percent of the total left ventricle was similar in the ETC-216-treated and vehicle-treated groups (FIG. 16). Rabbits treated with 3 mg/kg ETC-216 developed smaller infarcts (p<0.05) expressed as a percent of the area at risk compared to rabbits treated with vehicle (FIG. 16). A reduction in myocardial infarct size (p<0.05) was observed when the data were expressed as a percent of the total left ventricle (FIG. 16).

No significant differences were noted with a dose of 1 mg/kg between ETC-216 and vehicle in the size of the AAR when expressed as the percent of the left ventricle (FIG. 16). At 1 mg/kg, no significant differences were noted between groups as a percent of AAR (FIG. 16) and in myocardial infarct size expressed as a percent of the total left ventricle (FIG. 16).

A summary of the data from each of the four acute treatment groups (i.e., 100, 10, 3 and 1 mg/kg) and their respective controls are shown in FIG. 16. The AAR of infarction was similar in each of the four groups. Among the four dosing regimens, infarct size, whether expressed as percent of risk region or percent of the left ventricle, compared to the respective controls was reduced with ETC-216 doses of 100, 10 and 3 mg/kg. In contrast, infarct size in the group of animals receiving 1 mg/kg did not differ from that observed in the respective vehicle-treated group.

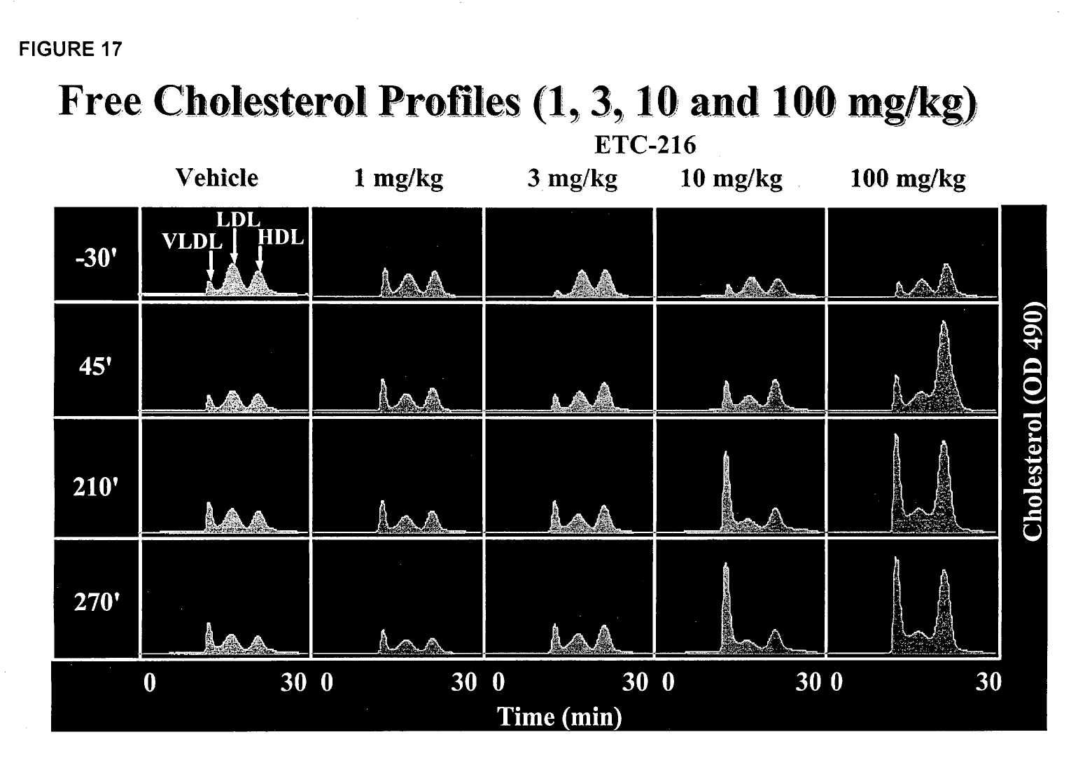

FIG. 17 shows examples of temporal changes in lipoprotein unesterified cholesterol. Blood samples were obtained from rabbits just prior to and periodically following administration of 1, 3, 10 or 100 mg/kg ETC-216 or vehicle. Shown are unesterified cholesterol profiles obtained in representative temporal blood serums samples where the serum lipoproteins were separated on the basis of size by gel-filtration chromatography with on-line unesterified cholesterol analysis. Note the rise in high density cholesterol unesterified cholesterol at 45 minutes after administration of ETC-216, especially at 100 mg/kg and to a lesser extent at 10 mg/kg despite the virtual absence of unesterified cholesterol in the intravenously administered ETC-216 test agent. Note also the delayed prominent rise in very low density lipoprotein unesterified cholesterol at 210 and 270 minutes following administration of either 10 mg/kg or 100 mg/kg ETC-216. Note also that changes in lipoprotein unesterified cholesterol were not apparent at the 3 mg/kg ETC-216 treatment dose at the time points assessed, however, this dose was cardioprotective as shown in FIG. 16.

The results demonstrate that 100 mg/kg, 10 mg/kg and 3 mg/kg doses are effective prophylactic doses of ETC-216.

### 6.4. Example 4

### ETC-216 Prevents Ischemia-Reperfusion Injury when Administered after the Onset of LAD Occlusion in the Occluded-Reperfused Rabbit Heart

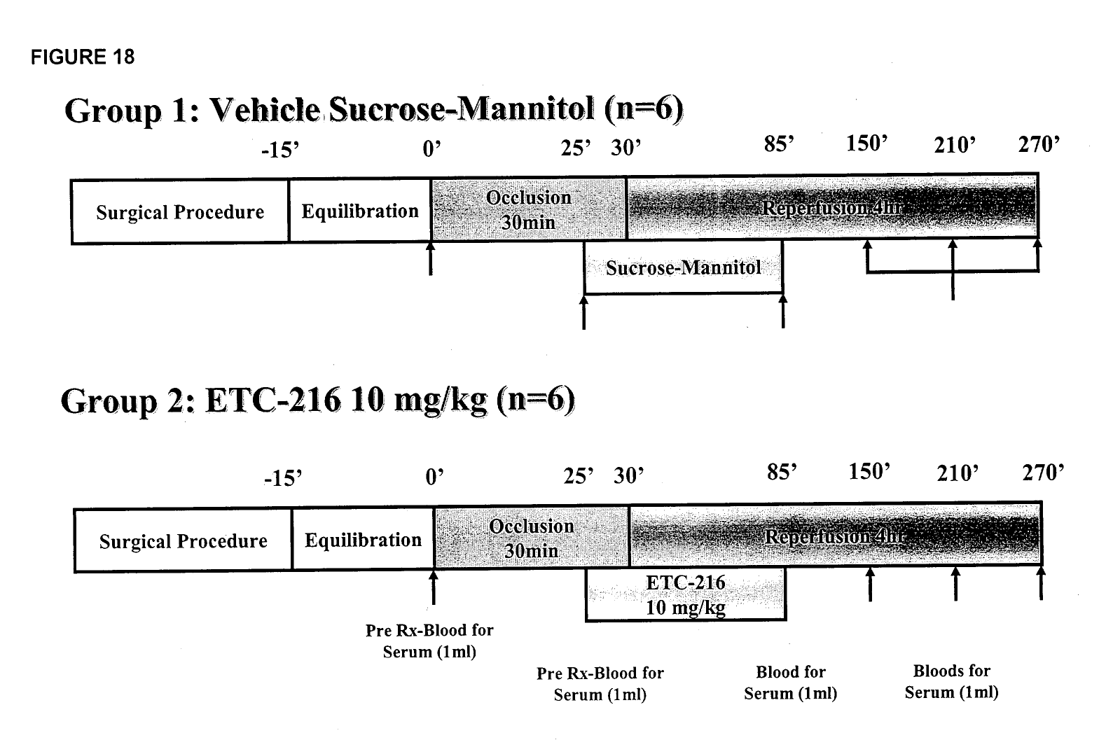

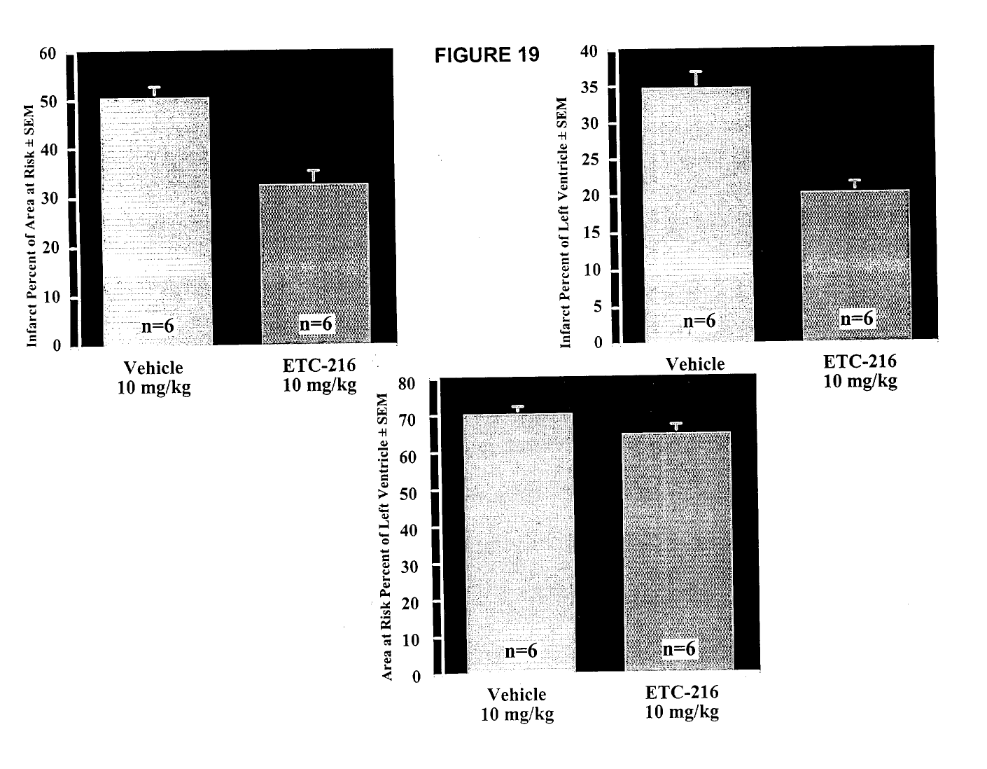

This example demonstrates the efficacy of ETC-216 in preventing or reducing ischemic reperfusion injury when administered after the ischemic or occlusive event. The studies in Examples 2 and 3 illustrate the prophylactic benefit of treating the heart muscle prior to the onset of ischemia. Therefore to determine if ETC-216 could protect the heart muscle after the onset of ischemia, the LAD was occluded prior to the administration of the test agent or vehicle. In this protocol, ETC-216 was tested as a single treatment in which the heart was exposed to 10 mg/kg of the agent or an equivalent volume of vehicle administered during the last 5 minutes of regional ischemia and continued through the first 55 minutes of reperfusion (FIG. 18). The AAR or ischemic region expressed as a percent of the total left ventricle for the 10 mg/kg treatment group was similar in the control group (FIG. 19). Rabbits treated with ETC-216 developed smaller infarcts (p<0.001) expressed as a percent of the AAR compared to rabbits treated with vehicle (FIG. 19). A reduction in myocardial infarct size (p<0.0005) also was observed when the data were expressed a percent of the total left ventricle (FIG. 19).

This example demonstrates that a single treatment administered after an ischemic event, mitigated or decreased ischemic reperfusion injury.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below.

All references cited herein are hereby incorporated by reference in their entireties.

We claim:

1. A method to treat, prevent or reduce ischemic reperfusion injury in a tissue or organ comprising contacting the tissue or organ with an effective amount of an apolipoprotein.

2. The method of claim 1 wherein the apolipoprotein is not a thiol containing apolipoprotein.

3. The method of claim 1 wherein the apolipoprotein is a thiol containing apolipoprotein.

4. The method of claim 1 wherein the apolipoprotein is apoA-I, apoA-II, apoA-IV, apoA-V, apoE or a variant or fragment thereof.

5. The method of claim 1 wherein the apolipoprotein is of human or non-human origin.

6. The method of claim 1 wherein the apolipoprotein is a natural or synthetic apolipoprotein, or a variant or fragment thereof.

7. The method of claim 1 wherein the apolipoprotein is a homogeneous mixture of apolipoproteins.

8. The method of claim 1 wherein the apolipoprotein is a heterogenous mixture of apolipoproteins.

9. The method of claim 1 wherein the apolipoprotein is a full length apolipoprotein, a fragment of a natural or a synthetic apolipoprotein, or a variant thereof.

10. The method of claim 1 wherein the apolipoprotein is apolipoprotein A-I, apolipoprotein A-I Milano or apolipoprotein A-I Paris.

11. The method of claim 10 wherein the apolipoprotein is apolipoprotein A-I Milano.

12. The method of claim 1 wherein the apolipoprotein is in the form of a complex comprising the apolipoprotein and a lipid.

13. The method of claim 12 wherein the lipid comprises one or more of a phospholipid, cholesterol, a triglyceride and a cholesterol ester.

14. The method of claim 13 wherein the phospholipid is selected from the group consisting of small alkyl chain phospholipids, phosphatidylcholine, egg phosphatidylcholine, soybean phosphatidylcholine, dipalmitoylphosphatidylcholine, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, (1,3)-D-mannosyl-(1,3)diglyceride, aminophenylglycoside, 3-cholesteryl-6′-(glycosylthio)hexyl ether glycolipids, cholesterol and cholesterol derivatives.

15. The method of claim 13 wherein the phospholipid is a phosphatidylcholine or an analogue thereof.

16. The method of claim 15 wherein the phospholipid is 1-palmitoyl-2-oleoyl phosphatidylcholine.

17. The method of claim 12 whereby the lipid and apolipoprotein form a liposomal structure.

18. The method of claim 1 wherein the apolipoprotein reduces tissue or organ oxidized products.

19. The method of claim 1 wherein the apolipoprotein reduces tissue or organ creatine kinase.

20. The method of claim 1 wherein the method is a therapeutic treatment.

21. The method of claim 1 wherein the method is prophylactic or preventative.

22. The method of claim 1 wherein the method reduces ischemic reperfusion injury.

23. The method of claim 1 wherein the tissue or organ is in an individual.

24. The method of claim 23 wherein the ischemic reperfusion injury is due to myocardial infarction, stenosis, at least one blood clot, stroke, intermittent claudication, peripheral arterial disease, acute coronary syndrome, cardiovascular disease or muscle damage as a result of occlusion of a blood vessel.

25. The method of claim 1 wherein the tissue or organ is extracorporeal.

26. The method of claim 25 wherein the tissue or organ is a transplant tissue or organ.

27. The method of claim 26 wherein the apolipoprotein is contacted with the transplant tissue or organ during transit.

28. The method of claim 26 wherein the apolipoprotein is contacted with the transplant tissue during transplantation.

29. The method of claim 24 wherein the apolipoprotein is contacted with the tissue or organ acutely after ischemia.

30. The method of claim 23 wherein the ischemic reperfusion injury is due to surgery of an individual and contacting the tissue or organ comprises administering a pharmaceutical composition comprising an apolipoprotein to the individual.

31. The method of claim 30 wherein the surgery is cardiac surgery.

32. The method of claim 31 wherein the apolipoprotein is administered during cardiac surgery.

33. The method of claim 30 wherein the cardiac surgery is coronary artery bypass surgery or percutaneous transluminal coronary angiography.

34. The method of claim 23 wherein the need for coronary artery bypass surgery is reduced.

35. The method of claim 23 wherein the need for percutaneous transluminal coronary angiography is reduced.

36. The method of claim 30 wherein the surgical recovery time is reduced.

37. The method of claim 23 wherein the stenosis is caused by one or more diseased blood vessels.

38. The method of claim 23 wherein the stenosis is mechanically induced by occluding one or more blood vessels.

39. The method of claim 23 wherein the injury is caused by one or more blood clots.

40. The method of claim 39 wherein the blood clot is caused by a plaque rupture.

41. The method of claim 1 wherein the injury is to a muscle.

42. The method of claim 41 wherein the muscle is cardiac muscle.

43. The method of claim 41 wherein the muscle is skeletal muscle.

44. The method of claim 41 wherein the muscle is smooth muscle.

45. The method of claim 1 wherein the injury is to an organ.

46. The method of claim 45 wherein the organ is heart, lung, kidney, spleen, liver or brain.

47. The method of claim 1 wherein the apolipoprotein is in the form of a 1:1 ratio of Apolipoprotein A-I Milano and 1-palmitoyl-2-oleoyl phosphatidylcholine.

48. The method of claim 11 wherein the apolipoprotein is administered parenterally.

49. The method of claim 23 wherein the apolipoprotein is administered intravenously, intraarterially, pericardially, perivascularly or into the coronary arteries.

50. The method of claim 1 further comprising administering a thrombolytic agent.

51. The method of claim 50 wherein the thrombolytic agent is tissue plasminogen activator (TPA), streptokinase, anistreplase, reteplase or urokinase.

52. The method of claim 1 further comprising administering an anticogulant or antiplatelet agent.

53. The method of claim 53 wherein the agent is aspirin, clopidogrel or heparin.

54. A method to prevent or treat a condition associated with oxygen deprivation followed by increased oxygen supply to a tissue or organ in need thereof comprising contacting the tissue or organ with an effective amount of an apolipoprotein.

55. The method of claim 54 wherein the condition associated with oxygen deprivation is neutrophil activation.

56. The method of claim 54 wherein the condition associated with oxygen deprivation is myeloperoxidase production.

57. The method of claim 54 wherein the method reduces the severity of the condition associated with oxygen deprivation.

58. The method of claim 54 wherein the apolipoprotein is contacted with the tissue or organ acutely after oxygen deprivation.
